(19) United States
(12) Patent Application Publication
(10) Pub. No.: US 2011/0143373 A1
(43) Pub. Date: Jun. 16, 2011

(54) METHOD OF EVALUATING THE INTEGRITY OF THE PLASMA MEMBRANE OF CELLS BY DETECTING GLYCANS FOUND ONLY INTRACELLULARLY

(75) Inventors: Tia Hirvonen, Vantaa (FI); Annika Kotovuori, Vantaa (FI); Virve Pitkanen, Helsinki (FI); Suvi Natunen, Vantaa (FI); Johanna Nystedt, Helsinki (FI); Sari Tiitinen, Vantaa (FI); Leena Valmu, Helsinki (FI); Jari Natunen, Vantaa (FI)

(73) Assignees: SUOMEN PUNAINEN RISTI, VERIPALVELU, Helsinki (FI); GLYKOS FINLAND LTD, Helsinki (FI)

(21) Appl. No.: 13/054,632

(22) PCT Filed: Jul. 16, 2009
(86) PCT No.: PCT/FI09/50627

§ 371 Date: Feb. 9, 2011

Related U.S. Application Data
(60) Provisional application No. 61/081,748, filed on Jul. 18, 2008.

(30) Foreign Application Priority Data
Apr. 16, 2009 (FI) 20095418

(51) Int. Cl. G01N 33/68; C12N 5/071; C12N 5/0775

(52) U.S. Cl. 435/7.21; 435/366

(57) ABSTRACT

The invention describes novel reagents that can be applied for analysis of the quality of human cells. The method evaluates the integrity of the plasma membrane of the cells by detecting novel glyco structures found only intracellularly. The method can be applied, for example, to demonstrate exposure of therapeutic cell preparation to potentially harmful conditions. It can also be used as a quality control tool in methods in which intact cell membrane is essential and it can be applied in separation of damaged cells from non- damaged.

The invention describes novel reagents that can be applied for analysis of the quality of human cells. The method evaluates the integrity of the plasma membrane of the cells by detecting novel glyco structures found only intracellularly. The method can be applied, for example, to demonstrate exposure of therapeutic cell preparation to potentially harmful conditions. It can be also used as a quality control tool in methods in which intact cell membrane is essential and it can be applied in separation of damaged cells from non-damaged.

### BACKGROUND OF THE INVENTION

The present invention reveals that certain glyco structures are found predominantly or solely in damaged cells, more specifically in cells whose membranes are damaged and hence resulting in exposure of epitopes which are not present on the surface of non-damaged cells. A preferred type of damage to the cells is necrotic cell damage. A preferred cell type in the present invention is human stem cell.

The inventors have revealed multiple carbohydrate markers on surface of human cells including stem cells, described in a recent international application, PCT/FI2008/050019. Some of the binders described in the application labeled only a fraction or subpopulation of cells. The co-pending application does not disclose present methods for analysis or production of damaged cells.

The quality of cell population, that is, the cells must be e.g. living, intact and able to divide, is a crucial factor for effective and safe therapeutic use of cells. There is no single useful method to evaluate all quality aspects of cell preparation but different methods must be used for different cell types and for different applications and indications. The present invention describes a novel method to evaluate the quality status of cellular preparations. In the invention, use of novel glyco structures detectable only in cells whose plasma membrane is damaged and particular binders for their detection are described. It is further demonstrated that their presence is associated with necrosis, the death, of cells. These glycans are said to be “cryptic”, i.e. they are not found or are non-accessible on the cell surface.

The complex carbohydrates present on glycolipids or glycoproteins are considered as cell surface components, they are synthesized in the Golgi apparatus and transported to the plasma membrane. A very limited set of glycan types have been shown to be associated with lysosomes, an intracellular organelle. Methods for targeting or labeling lysosomal glycans by specific binder reagents have not been generally established. The present invention is directed to multiple glycan types clearly different to the known lysosome-associated glycosylation, such as mannose-6-phosphate containing N-glycans. In a preferred embodiment of present invention, the novel cryptic epitopes are compared to intracellular organelle-specific glycoepitopes, such as lysosomal glycosylation.

It is known that certain glycans, such as certain globoseries glycolipids, can be cryptic, i.e. non-accessible, on the cell surface. The cell labeling methods of Lampio et al. (Eur J Biochem 1986 157, 611-6) using periodic acid are directed to the cell surfaces of intact erythrocytes, a cell type clearly different from stem cells of the present invention. This crypticity is not well known and is considered to result from specific interactions between cell surface molecules. The present invention reveals novel cryptic glycan epitopes present in damaged cells, including permeabilized or necrotic cells. In a preferred embodiment the glycan epitopes of the present invention are compared to known glycosylation of cell surface or plasma membrane. Such glycans can be labelled with periodic acid on intact cells.

Lysosomal dysfunction and the accumulation of gangliosides GM3, GD1b and GT1b has been observed with increased senescence associated with increased lysosomal β-galactosidase activity, decreased proliferation, and increased cell size in certain endothelial cells exposed to glycated collagen I (Patschan, S. et al. Am J Renal Physiol 2008, F100-9). The phenomenon appears different from necrosis according to the invention, especially under the specific conditions according of the invention. Plasma membrane damage or permeabilization, which would reveal marker structures was not indicated in the publication. It is further realized that other cryptic glycan epitopes according to the invention are products of different biosynthetic pathways and clearly different from gangliosides of Paschan et al. Further, as glycosylation is cell type specific, no data on endothelial cell lines (Paschan et al) nor those on bronchial gland cell (Castells M T et al. J. Histochem Cytochem 1992) are relevant to the preferred cells of the present invention.

A lectin labeling study certain of cell types has indicated labeling of intracellular material, but there is no indication of labeling with high specificity glycan binders, such as antibodies (Franz et al. 2006, Cytometry A, 69:230-239). The cells used in the study were also different from the cells of the present invention. Again, due to the cell type specificity of glycosylation, the lectin binding results cannot be generalized. The actual exact target glycan structures cannot be deduced based solely on the lectin binding experiment.

It is realized that there are cryptic epitopes, which may be revealed by in vitro protease treatment. The cryptic glycan markers of the present invention are not exposed by trypsin treatment to significant extent, the markers were practically not observed in trypsin-treated cells.

### BRIEF DESCRIPTION OF THE DRAWINGS

FIG. 1. Flow cytometry analysis with anti-glycan antibodies of bone marrow (BM) and cord blood (CB) mesenchymal stem cells (MSC) and osteogenically differentiated cells (OG) detached from culture plates either by 2 mM EDTA for 15 min (MSC EDTA), 10 mM EDTA for 30 min (CB OG EDTA) or 1 h (BM OG EDTA) or 0.25% trypsin for 3 min (trypsin). Histogram overlays are shown with staining antibodies in red fill and secondary controls in grey fill. The percentage of positive cells was determined as the cell population with more fluorescence intensity than 99.5% of cells stained with secondary antibody only. ND=not determined. * 71.3% and 2.7% of cells were positive for anti-Tn B1.1 and anti-sialyl Tn B35.1, respectively, at week 3 of differentiation; 14.8% and 1.8% of cells were positive for B1.1 and B35.1, respectively, at week 4. The histograms depict fully differentiated cells at week 6. The antibodies detected novel ‘cryptic’ subpopulations in EDTA-treated but not in trypsin-treated populations.

FIG. 2. Flow cytometry analysis of bone marrow mesenchymal stem cells treated with 2 mM EDTA for 15 min, 30 min, 1 h or 2 h, and stained with secondary antibody only (upper row) or anti-H-type 2 blood group antigen (middle row), x-axis; and propidium iodide, y-axis. Propidium iodide positive cells are in the upper left quadrant of the dot plot, cells positive for antibody staining are in the lower right quandrant, and double positive cells are in the upper right quadrant. The percentage of positive cells was determined as the cell population with more fluorescence intensity than 99.5% of cells stained with secondary antibody only without propidium iodide. The bottom row shows histogram overlays with staining antibody (red fill) and secondary control (grey fill).

FIG. 3. Bone marrow mesenchymal stem cells were detached from culture plates with either 2 mM EDTA (first row) or trypsin (all the other rows), and incubated at +56° C. for 30 min (third row) or with 0.1% Triton X-100 for 10 min at room temperature (bottom row). The cells were stained with propidium iodide (left column), a Fab recognizing a sialidase sensitive epitope (Fab1.4.24; middle column) or anti-Lewis a (right column), and analyzed by flow cytometry. The intensity of propidium iodide staining is depicted on the x-axis of the dot plots. For the antibody stainings histogram overlays are shown with staining antibodies in red fill and secondary controls in grey fill. The percentage of positive cells was determined as the cell population with more fluorescence intensity than 99.5% of cells stained with secondary antibody only.

FIG. 4. a) Bone marrow mesenchymal stem cells were labeled with anti-Tn and sorted by FACS. b) The sorted subpopulations were plated separately and let to attach and proliferate for 3 days.

FIG. 5. Glycan binding specificity of mAb 3C96 (anti-GT1b). Schematic representations of the glycan structures are shown for the best binders. Symbols: light circle: galactose, dark circle: mannose, dark square: N-acetylglucosamine, light square: N-acetylgalactosamine, triangle: fucose, SO₃: sulphate, horizontal line: β1,4-linkage, vertical line: α1,2-linkage, rising diagonal line: β1,3-linkage, declining diagonal line: β1,6-linkage.

FIG. 6. Glycan binding specificity of mAb Tra-1-81. Schematic representations of the glycan structures are shown for the best binders. Symbols: light circle: galactose, dark circle: glucose, dark square: N-acetylglucosamine, light square: N-acetylgalactosamine, triangle: fucose, horizontal line: β1,4-linkage, vertical line: α1,2-linkage, rising diagonal line: β1,3-linkage, declining diagonal line: β1,6-linkage.

FIG. 7. Glycan binding specificity of mAb Tra-1-60. Schematic representations of the glycan structures are shown for the best binders. Symbols: light circle: galactose, dark circle: glucose, square: N-acetylglucosamine, horizontal line: β1,4-linkage, vertical line: α1,2-linkage, rising diagonal line: β1,3-linkage, declining diagonal line: β1,6-linkage.

FIG. 8. Glycan binding specificity of mAb B1.1 (anti-Tn). Schematic representations of the glycan structures are shown for the best binders. Symbols: diamond: N-acetylneuraminic acid, light circle: galactose, dark circle: mannose/at the reducing terminus glucose, dark square: N-acetylglucosamine, light square: N-acetylgalactosamine, triangle: fucose, SO₃: sulphate, horizontal line: β1,4-linkage, vertical line: α1,2-linkage, rising diagonal line: β1,3-linkage (for sialic acid α2,3-linkage), declining diagonal line: β1,6-linkage.

FIG. 9. Glycan binding specificity of Fab1.4.24 Schematic representations of the glycan structures are shown for the best binders. Symbols: diamond: N-acetylneuraminic acid, light circle: galactose, dark circle: mannose, dark square: N-acetylglucosamine, light square: N-acetylgalactosamine, triangle: fucose, SO₃: sulphate, horizontal line: β1,4-linkage, vertical line: α1,2-linkage, rising diagonal line: β1,3-linkage (for sialic acid α2,3-linkage), declining diagonal line: β1,6-linkage.

FIG. 10. Glycan binding specificity of mAb KM231 (anti-sialyl Lewis a). Schematic representations of the glycan structures are shown for the best binders. Symbols: dark diamond: N-acetylneuraminic acid, light diamond: N-glycolylneuraminic acid, light circle: galactose, dark circle: glucose, dark square: N-acetylglucosamine, light square: N-acetylgalactosamine, triangle: fucose, SO₃: sulphate, horizontal line: β1,4-linkage, vertical line: α1,2-linkage, rising diagonal line: β1,3-linkage (for sialic acid α2,3-linkage), declining diagonal line: β1,6-linkage.

### DESCRIPTION OF THE INVENTION

The present invention revealed that there are specific cryptic glycan epitopes in cells, which can be observed in cells after exposure to harmful or potentially harmful conditions and that these markers can be applied in the quality evaluation of cellular preparation, for example, in the context of cell therapy or in other methods in which intact cell membrane is essential. The “cryptic glycan epitope” (also “cryptic epitope”) means a glycan structure, which is essentially non-detectable on a native, intact cells, but which is detectable in the cells that have been exposed to harmful or cell damaging conditions. Such conditions can be membrane damaging or permeabilizing conditions. The invention is especially directed to a method of targeting or identifying damaged cell population or to methods for estimation the proportion of cells damaged. In the present invention, the damaged cells are identified by subjecting them to a specific glycan binder reagent which detects one or several cryptic glycan epitopes. Invention is directed to a method of targeting of a cell population, involving a step of binding a specific glycan binder reagent of the invention to a cryptic glycan epitope in cells, wherein the cryptic glycan epitope is exposed under cell membrane damaging or harming conditions and wherein the glycan binder does not bind substantially on intact cells. Targeting means here evaluation, quality control or inspection of the quality of cells, or manipulation of a cell population, preferably by sorting or selecting cells or binding to immobilized binders.

Stem cell preparations intended for therapeutic use, as well as many other cell preparations, often undergo various handling processes such as freezing and thawing or detachment from culture plates. These processes may cause physical stress that damages the cells so that the integrity of the cell membrane becomes compromised and some of the cells undergo necrosis. The necrotic cells cannot be assumed to be effective in therapeutic use, in fact, they can be even harmful as they may induce the immune response against intracellular molecules.

The invention is in one embodiment directed to detection of necrosis or cell membrane damage during regular cell handling and culture. These conditions may include chelating agents, lipids similar to detergents, or damaging natural physical or temperature conditions. In a separate embodiment the cell handling also includes physiological conditions wherein cells are grown in vivo or ex vivo. Furthermore, it is realized that, for example, for the manipulation of cells or their surface structures, some toxic agents must be used resulting in exposure of the cryptic epitopes and their detection can be used to estimate the degree of damage induced by the treatment.

The invention is directed to the detection of membrane damage as a part of the quality control process for therapeutic cell preparations, for example in combination with antibodies used for the validation of differentiation state.

The binder reagent used for the detection of the cryptic epitope is preferably a macromolecule, such as a poly- or oligopeptide or whole protein, which binds specifically or more preferably exclusively to the glycan structures. Preferred protein binder molecules include antibodies, lectins and glycan binding or modifying enzymes and enzymatically non-active mutants thereof.

The invention is directed to the detection of the cryptic carbohydrate markers which are revealed after exposure to conditions causing necrosis or damage to cell membranes. The invention is in a preferred embodiment directed to the detection or evaluation of cells exposed to any cell harming and/or damaging conditions causing the cryptic glycan epitopes to be available for recognition by binder reagent(s).

It is realized that many different condition can result in detection of the cryptic epitopes. They can include, for example exposure of cells to (i) a toxic agent, preferably a cation chelating agent, more preferably a divalent cation chelating agent, most preferably Ethylenediamine tetra-acetic acid (EDTA), or (ii) lipophilic agents, preferably detergents, more preferably Triton X-100; or iii) to harmful temperature, or iv) altered gas conditions such as oxygen, or carbondioxide conditions, or any combinations of i-iv. Harmful temperature conditions include exposing cells to heat above or below the regular mammalian or human cell culture conditions with the temperature of about 35° C.-37° C. Preferred heating conditions include above 38 or below 35° C. It is realized that the time of exposure or concentrations used for toxic agents can vary. The invention can be applied for optimization of temperature stress, or any other stress that human stem cells can survive without necrosis.

The invention is further directed to cell handling and/or toxic conditions when the epitopes are revealed in large part of cell population, not regularly observable after regular mesenchymal stem cell/mesenchymal cell such as detachment processes by chelating agent preferably EDTA (as described for individual glycan markers and antibody clones and respective cell types in the reference PCT/FI2008/050019), preferably including long exposure to EDTA according to the invention such as more than 0.5 hours, more preferably over 40 minutes, even more preferably 1 hour or more such as at least 2 hours and/or exposure to EDTA concentrations over 2.1 mM, more preferably at least 3 mM or more preferably at least 5 mM or most preferably at least 10 mM. It is realized that there is no indication of the structures from various preferred cell types under cell permeabilization conditions or as a practically uniformly (expressed at least in 85%, or 90%, or 95%, or most preferably 98% of cells) expressed intracellular marker, in a preferred embodiment the cells are non-cancerous cell, preferably stem cells. The invention is in a separate embodiment directed to major exposure to cell permeabilizing conditions such as membrane permeabilization by detergents causing exposure of cryptic epitopes in most or practically all cells, preferred conditions for this include the use of lipophilic agents, preferably detergents, or heat treatment at high temperature such as at 56 degrees of Celsius or between 51-65 more preferably between 45-80, most preferably above 40 and under 100 degrees of Celsius, depending on stability of the cells. In a preferred embodiment the invention is directed to the analysis of cells when the cells have been exposed to lipophilic agents. The preferred lipophilic agents are detergents, most preferably Triton X-100 type of detergents. In a preferred embodiment the cells are treated with the lipophilic agent in order to induce the availability of the cryptic glycan epitope for the binder reagent. More preferably the lipophilic agent is used in a sufficient amount to cause the majority or practically all of the cells to reveal the cryptic epitope(s). In a preferred embodiment the lipophilic agent is used in an sufficient amount to allow the detection of the epitope in the majority or practically all of the cells while still “maintaining the integrity” of cells so that they can be analyzed. It is realized that exposure of the novel cryptic glycan epitope and maintaining the integrity of cells is not trivial task as lipophilic agents have tendency to release glycoproteins and lipids from cells and lyse the cells and on the other hand the amount should be large enough to cause the cryptic epitope to be revealed, more preferably in the majority of the cells or even more preferably in practically all of the cells. In a preferred embodiment the invention is directed to the use of detergent in an amount equivalent to 0.1% Triton X-100 for 10 min at room temperature to expose the novel cryptic glycan epitopes while maintaining the integrity of the cells so that they can be analyzed, preferably flow cytometry analysis. The major exposure is in a preferred embodiment used as a reference for the spontaneous necrosis during regular cell handling conditions intended not to permeabilize or cause necrosis or kill cells. In a preferred embodiment the cell permeabilization conditions are used as control for cryptic epitope exposure under natural type conditions. The permeabilization condition expose the novel cryptic glycan epitopes while maintaining the integrity of the cells so that they can be analyzed, preferably flow cytometry analysis.

It is further realized that differences in the nature of cell populations affect the conditions for exposure of the novel cryptic epitopes and the invention is directed to the optimization of the conditions for a specific cell population or part of it by testing multiple conditions and selecting the optimal conditions for the specific cell population or subpopulation.

The invention is further directed to the use of the present method with other methods analyzing the degree of damage in cells, especially permeabilization or damage of cell membranes. It is realized that the other methods are based on different biochemical targets and likely, at least in part, measure different aspects of cellular integrity, such as different levels of membrane permeabilization or major permeabilization or reorganization of different cellular organs.

The preferred cell types according to the invention include mesenchymal stem cells and cells differentiated thereof. Mesenchymal stem cells can be derived from many tissues, such as blood bone marrow or cord blood. The differentiated cells include, among many others known in the scientific literature, mesenchymal cells differentiated to osteogenic, adipogenic or chondrogenic directions. The differentiated cells are in a preferred embodiment derived from ex vivo cell culture, and are preferably non-manipulated cells and in a preferred embodiment modulated cells. In a preferred embodiment the mesenchymal cells are evaluated for the presence of the cryptic epitopes on native cells and/or damaged cells.

In a preferred embodiment the invention is directed to the evaluation of mesenchymal cells as a quality control step in a cell production process.

The invention is further directed to a method to evaluate the presence of the novel target saccharides as cryptic exposable elements in other eukaryotic cells, preferably in mammalian cells. The human cells may include cells in the context of pathogenesis such as autoimmunity (such as type I diabetes, rheumatoid arthritis or autoimmune thyroiditis), inflammation (such as inflammatory bowel disease or infections) or malignancy (leukemias or solid cancers). They also can be other stem cells such as embryonic stem cells, induced plutipotent stem cells (iPS), or hematopoietic stem cells, or cells and tissues derived thereof. In a preferred embodiment the other cells are evaluated for the presence of the cryptic epitopes on native cells and damaged cells. In one embodiment, the present invention is used in context of evaluating the toxicity for stem cells or tissues, in particular those derived from embryonic stem cells or from iPS cells, of compounds screened for potential drug development. In this embodiment, human stem cells induced to differentiate in vitro to, for example, liver, heart or brain tissues, can be used for screening effect of novel molecules for potential drugs in tissues of human origin.

The invention is in a preferred embodiment directed to “natural-type conditions” revealing the cryptic glycan epitope in at least part of cells, including regular cell handling in culture conditions and cell culture or sample derived from in vivo physiological conditions, wherein the handling does not include exposure to significant EDTA concentration (such as over 1 nM or preferably over 10 microM). The cryptic glycan epitope is preferably revealed in a substantial part of cells, more preferably in at least 1%, even more preferably in at least 3% of cells but less than 90%, more preferably less than 60% and most preferably as minor population of less than 30% of cells of a cell population or preparation. The cryptic glycan epitopes were originally observed as a minor population under regular cell handling conditions, including exposure to EDTA. The natural type conditions may include natural type cell damaging conditions according to the specification of the invention, preferably including natural molecules (natural chelating agents, lipids similar to detergents or modest temperature), or damaging natural physical/temperature conditions and/or substantial amount (such as over 1 nM or preferably over 100 nM) of Triton-X100 or preferably all synthetic detergents and/or excluding temperatures over 43 degrees of Celsius more preferably not 42 degrees of Celsius, most preferably excluding all of these conditions.

It is realized that cryptic epitopes would occur also in context of limited exposure to other harmful or natural-like conditions.

The present invention is especially directed to a method for evaluating the presence of the novel cryptic epitopes in cells including following the steps:
1) Exposing a cell preparation or its fraction to conditions damaging the cell membranes and exposing the cryptic epitopes
2) Contacting the cell preparation with said binder reagent for the novel cryptic epitope after the exposure to the conditions damaging the cell membranes and exposing the cryptic epitopes
3) Measuring the binding of the binder molecule to the cryptic epitope.

In another preferred embodiment the invention is directed to measurement of the presence of the cryptic epitopes under non-damaging conditions and under the damaging conditions revealing cryptic epitopes. The preferred method includes the following steps:
1) Contacting a cell preparation or fraction of it with the binder reagent for the novel cryptic epitope under native cell conditions, more specifically under conditions not substantially damaging the cell membranes and exposing the cryptic epitopes.
2) Measuring the binding of the binder molecule to the cryptic epitope.
3) Exposing a cell preparation or its fraction to conditions damaging the cell membranes and exposing the cryptic epitopes
4) Contacting the cell preparation with said binder reagent for the novel cryptic epitope after the exposure to the conditions damaging the cell membranes and exposing the cryptic epitopes
5) Measuring the binding of the binder molecule to the cryptic epitope.

It is realized that it would be useful to prepare two fractions of the same cell preparation and perform the binding measurements to the different cell fractions. Otherwise the conditions should be adjusted so that the binder reagent from the first measurement would not harm the second measurement. In an embodiment the characterization assay measures both exposed and cryptic structures and the results are compared. In an embodiment the comparison is performed between the marker on native cells as percent of positively labeled cells and the marker on potentially damaged cells, for example after a treatment with some reagents, as percent of positively labelled cells. The proportion of labelled cells can be measured by various detection methods described in literature, for example by using fluorescence detection by flow cytometry.

The invention is further directed to the use of the “method(s) to evaluate the presence of the novel cryptic epitope”, wherein cells, which are known to contain cryptic epitopes are exposed to novel conditions or reagents suspected to cause damage, that is, exposure of the cryptic epitopes. The cells are exposed to the novel condition or reagents and the level of labeling of the cell population with specific binder reagent(s) is measured, and result is preferably compared with control cells treated under conditions known to cause the revealing of cryptic epitopes. In a preferred embodiment cells containing the cryptic epitopes are used for the screening of toxic agents, lipids or other molecules, such as drugs, poisons, pharmaceuticals, environmental chemicals etc. suspected to cause revealing of the cryptic glycan epitope(s).

In another preferred embodiment the invention is directed to the use of the binder reagents for cryptic epitopes in arrays comprising more than one binder reagent. It is realized, that the reagents have different specificities and due to different biosynthetic pathways the expression of the different cryptic glycan epitopes varies in cells and therefore their use in combinations is especially preferred. The binders of present invention are preferably used together with other binding molecules for the target cells, preferably in arrays. The other binder reagents include preferably at least one other binder for available, non-cryptic, cell surface structure, preferably other binders for at least two non-cryptic cell surface glycan epitopes, and even more preferably binders for at least three non-cryptic cell surface glycan epitopes. The other binder reagents further include preferably at least one specific other binder reagent, which does not bind to cryptic or non-cryptic glycan epitopes of the cells. In a preferred embodiment the array is an immobilized binder array, more preferably an antibody array.

In a preferred embodiment the cells comprising exposed cryptic epitopes are sorted or isolated from a cell population, which comprises cells comprising exposed and cells comprising non-exposed cryptic epitopes. It is realized that it is especially useful to sort damaged necrotic cells having exposed cryptic epitopes from living cells that do not have exposed cryptic epitopes. The invention is further directed to cell populations which are sorted, preferably by a specific binder reagent to contain mainly cells enriched with cryptic epitopes and in another embodiment “live cells”, meaning cell population containing only cells, which do not have their cryptic epitopes exposed.

The invention is further directed to the culture of the “live cells” population. It was shown that this population can be cultured. The isolated live cells are especially directed to the optimization of cell culture processes for the production of cells, especially mesenchymal cells according to the invention. The invention is further directed to the use of the live cells for in vivo uses, such as in vivo therapeutic uses including therapy development and optimization.

The invention is in an embodiment directed to the release of the binders from the cryptic epitopes. This can be done using several methods including: a) release of cells from soluble binders after enrichment or isolation of cells by a method involving a binder, b) release from solid phase bound binders after enrichment or isolation of cells or during cell culture e.g. for passaging of the cells

Preferred Structures of Novel Cryptic Glycan Markers

The novel cryptic glycan markers for mesenchymal stem cells include (Tables 1-7)
Type I N-acetyllactosamines comprising the core epitope Galβ1-3GlcNAc and its fucosylated and/or sialylated derivatives,
Epitope Galβ1-4GlcNAc and its fucosylated and/or sialylated and/or sulphated derivatives,
Lewis a, sialyl-Lewis a, or blood group A epitopes,
Larger multisialylated ganglioseries gangliosides,
Type II N-acetyllactosamines including H-type II antigen and keratan sulphate epitopes,
Small mucin-type O-glycans including Tn and sialyl Tn antigens.

The invention is further directed to the types of structures recognized by the Fab-fragment Fab1.4.24. (Table 5). The invention is directed to antibody clones effectively recognizing the cryptic glycan epitopes, more preferably to anti-Lewis a (Table 6), anti-GT1b (Table 1), anti-Tra-1-81 (Table 2), anti-Tra-1-60 (Table 3), anti-Tn (Table 4). Most preferably the specificity is essentially the same when measured for binding against multiple glycan epitopes.

The cryptic glycan marker structures comprise common core Galβ3/4HexNAc or GalNAcα which may further include sialic acid and fucose and additional core structures. The invention revealed, see Table 7 and Table 8 (especially for EDTA/chelation conditions), that most antibodies bind type I and type II N-acetyllactosamine oligosaccharide sequences comprising core structure Galβ3/4GlcNAc, and sialylated or fucosylated or sulphated derivatives thereof, as indicated in Tables 1-7, more preferably including type I lactosamines preferably Galβ3GlcNAc or Lewis a or SAα3Galβ3GlcNAc or sialyl-Lewis a, more preferably terminal non-reducing end Galβ3GlcNAc and/or Lewis a, or separately preferred type II lactosamines such as oligosaccharide sequences Galβ4GlcNAc, or SAα3Galβ4GlcNAc or Lewis x or sialyl-Lewis x, or Fucα2Galβ4GlcNAc, more preferably H type II, Lewis x or sialyl-Lewis x. The preferred non-reducing end oligosaccharide sequences are according to Formula (SAα3)ₘGalβ3/4(Fucα4/3)ₙGlcNAc, wherein SA is sialic acid preferably Neu5Ac, m and n are 0 or 1 independently, in a specific embodiments m is 0 or n is 0; and in another both m and n are 1.

The invention also revealed a novel glycan binder antibody reagent Fab1.4.24 (or Fab1) with several unusual oligosaccharide and sialic acid linkage structure binding specificites and high specificity in recognition of cryptic glycan epitopes (Table 5). Low molecular weight antibody reagents including “single chain antibodies”, such as single chain Fvs, Fab fragments, Fab′ fragments, F(ab′) fragments, disulfide linked Fvs (sdFvs), Fvs, and fragments comprising or alternatively consisting of, either a VL or a VH domain, are preferred because of the effective recognition of cryptic epitopes similar to those detected by Fab1.4.24, and because of high accessibility to membrane surfaces by lower molecular weight antibody reagents. These can be produced from other antibodies by standard proteolysis and/or recombinant expression technologies.

The novel cryptic markers further include additional mesenchymal stem cell epitopes recognized by the specific antibody clones according to invention.

Preferred elongated cryptic glycan structures The preferred elongated cryptic glycan structures include the following oligosaccharide sequences. The invention is especially directed to high specificity binder reagents recognizing the preferred oligosaccharide sequences, preferably terminal oligosaccharide sequences. Most of the preferred elongated structures are glycolipid or poly-N-acetylactosamine type structures clearly different from e.g. from O-glycan core linked structures.

Preferred ganglioside structures The preferred ganglioside structures include structures recognizable by the monoclonal antibodies A2B5 and/or 3C96. The specificity of A2B52 [Saito Met al. Comp. Biochem. Biophys. Mol Biol. (2002) 131 (3) 433-41; Saito M et al. J Neurochem. (2001) 78 (1) 64-74; Kasai and Yu Brain Res. (1983) 277 (1) 155-8] includes especially c-series gangliosides, preferably:
GT3 with the glycan structure SAα8SAα8SAα3Galβ4Glc and more preferably with common gangliotetratetrasaccharide core structure,
GQ1c with the glycan structure SAα3Galβ3GalNAcβ4(SAα8SAα8SAα3)Galβ4Glc,
GP1c with the glycan structure SAα8SAα3Galβ3GalNAcβ4(SAα8SAα8SAα3)Galβ4Glc,
and hexasialyl ganglioside SAα8SAα8SAα3Galβ3GalNAcβ4(SAα8SAα8SAα3)Galβ4Glc, thus
   The preferred A2B5 target c-series gangliosides are according to the formula:

[SAα8]ₘ[SAα3]ₙ[Galβ3]ₚ[GalNAcβ4]_{q}(SAα8SAα8SAα3)Galβ4Glc,

wherein m is 0, 1 or 2; n, p and q are 0 or 1, independently, with the provisio that when q is 0 then also p, n, and m are 0; and when p is 0, then n, and m are 0; and when n is 0 then m is 0. In a preferred embodiment p and q are 1 and the gangliosides comprise the tetrasaccharide core.

SA is here sialic acid preferably Neu5Ac or Neu5Gc or natural derivative thereof such as acetylated, sulphated or lactylated variant thereof, more preferably Neu5Ac or Neu5Gc or O-acetylated variant thereof and more preferably Neu5Ac or Neu5Gc and most preferably Neu5Ac.

The specificity of GT1b 3C96 includes especially:
GT1b with glycan structure SAα3Galβ3GalNAcβ4(SAα8SAα3)Galβ4Glc

The invention is further directed to antibody target structures and antibodies binding specifically these, wherein the cryptic glycan target structure epitopes are included in target structures of both A2B5 and 3C96 specificites including
GT1b with glycan structure SAα3Galβ3GalNAcβ4(SAα8SAα3)Galβ4Glc
   and following gangliosides with the common tetrasaccharide core structure
[SAα8]ₘSAα3Galβ3GalNAcβ4([SAα8]ₙSAα8SAα3)Galβ4Glc,
   wherein m is 0, 1 or 2 and n is 0 or 1, independently.

Preferred N-acetyllactosamine structures are according to Formula [Mα]ₘGalβ1-3/4[Nα]ₙGlcNAcβxHex

wherein x is linkage position 2, or 3, wherein m, and n are integers 0, or 1, independently; and
Hex is Man and βxHex is β2Man, or Hex is Gal and βxHex is β3Gal
M and N are monosaccharide residues being i) independently nothing (free hydroxyl groups at the positions) and/or ii) SA which is Sialic acid linked to the 3-position of Gal and/or iii) Fuc (L-fucose) residue linked to the 2-position of Gal and/or 4 position of GlcNAc,
with the provision that when Gal is linked to the position 4 of GlcNAc, the N is nothing and M is
Fucα2 and the structure is H type II or M is SAα3/6, preferably SAα3 and the structure is keratan sulphate structure when Gal is linked to the position 3 of GlcNAc, and the structure is type I N-acetyllactosamine so that the N is nothing or Fucα4 and M is nothing or SAα3. When the structure is keratan sulphate epitope the 6-position of GlcNAc and/or Gal may be modified by sulphate ester residue.

Preferred elongated epitopes. It is realized that the exact structure on the reducing end of a glycan can be used for specific recognition of a glycan structure. It is known that high specificity antibodies can recognize the terminal epitope specifically on a specific reducing end core structure. The invention is especially directed to specific reducing end elongated variants of the glycan target structure present on the human cells and recognizable by specific antibodies against the cryptic epitopes. The preferred elongated core structures include:

1) N-acetyllactosamine/lactose core structures including:
a. Lacto-/neolacto glycolipid core structures including reducing structures and
b. poly-N-acetyllactosamine/keratan sulphate core structures.

2) N-glycan core structures comprising β-linkage to Manα3/6, more preferably β2-linkage to Manα3- and/or Manα6.

The preferred elongated structures observable on human mesenchymal stem cells are in Table I.

Preferred Type I N-Acetyllactosamine Structures

The preferred I N-acetyllactosamine structures are according to the Formula TI [SAα3]ₘGalβ3[Fucα4]ₙGlcNAcβ3Gal[β4Glc(NAc)ₚ]_{q}

wherein m, n, p and q are integers 0, or 1, independently SA is Sialic acid linked to 3-position of Gal and/or Fuc is L-fucose residue linked to 4 position of GlcNAc. The variable p and q indicate that when p is 1 than the epitope, which is recognized by the binder reagent includes at least part of the reducing end β4Glc or β4GlcNAc-structure. The invention is further directed to antibodies having specificity recognize at least part of 4Glcβ or β4GlcNAcβ-structures at the reducing end and the reducing end linkage structure. In a preferred embodiment the binder has same or similar specificity with the antibody clones used in examples.

The preferred elongated structures include: Lewis c/type I N-acetyllactosamine core structures Galβ3GlcNAcβ3Gal, Galβ3GlcNAcβ3Gal4, Galβ3GlcNAcβ3Galβ4Glc(NAc), Galβ3GlcNAcβ3Galβ4Glc, and Galβ3GlcNAcβ3Galβ4GlcNAc and Lewis a structures Galβ3(Fucα4)GlcNAcβ3Gal, Galβ3(Fucα4)GlcNAcβ3Galβ4, Galβ3(Fucα4)GlcNAcβ3Galβ4Glc(NAc), Galβ3(Fucα4)GlcNAcβ3Galβ4Glc, and Galβ3(Fucα4)GlcNAcβ3Galβ4GlcNAc and sialyl-Lewis a structures SAα3Galβ3(Fucα4)GlcNAcβ3Gal, SAα3Galβ3(Fucα4)GlcNAcβ3Galβ4, SAα3Galβ3(Fucα4)GlcNAcβ3Galβ4Glc(NAc), SAα3Galβ3(Fucα4)GlcNAcβ3Galβ4Glc, and SAα3Galβ3(Fucα4)GlcNAcβ3Galβ4GlcNAc

Preferred H Type II N-Acetyllactosamine Structures

The preferred elongated H type II structures include Fucα2Galβ1-4GlcNAcβxHex, wherein Hex is Man and βxHex is β2Man, or Hex is Gal and βxHex is β3Gal

The preferred H type II elongated on N-acetyllactosamine structures are according to the Formula TIIa Fucα2Galβ4GlcNAcβ3Gal[β4Glc(NAc)ₚ]_{q}

wherein m, and q are integers 0, or 1, independently and Fuc is L-fucose residue linked to 2 position of Gal. The variable p and q indicate that when p is 1 than the epitope, which is recognized by the binder reagent includes at least part of the reducing end β4Glc or β4GlcNAc-structure. The invention is further directed to antibodies having specificity recognize at least part of 4Glcβ or β4GlcNAcβ-structures at the reducing end and the reducing end linkage structure. In a preferred embodiment the binder has the same or similar specificity to the antibody clones used in examples.

The preferred glycolipid/polylactosamine structures include Fucα2Galβ4GlcNAcβ3Gal, Fucα2Galβ4GlcNAcβ3Galβ, Fucα2Galβ4GlcNAcβ3Galβ4, Fucα2Galβ4GlcNAcβ3Galβ4Glc(NAc), Fucα2Galβ4GlcNAcβ3Galβ4Glc, and Fucα2Galβ4GlcNAcβ3Galβ4GlcNAc.

Preferred N-Glycan H Type II Structures

Specific elongated H type II structure epitopes are especially expressed on N-glycans. Preferred H type II structures are β2-linked to biantennary N-glycan core structure, Fucα2Galβ4GlcNAcβ2Man and more preferably Fucα2Galβ4GlcNAcβ2Manα and even more preferably Fucα2Galβ4GlcNAcβ2Manα3/6Manβ4.

Preferred Small Mucin-Type O-Glycans Including Tn and Sialyl Tn Antigens

The preferred small mucin type O-glycans are according to the Formula (SAα6)ₙGalNAcα(Ser/Thr)ₘ
wherein n and m are 0 or 1, independently, and wherein SA is sialic acid according to the invention, preferably Neu5Ac. When n is 0, then the structure is Tn antigen with formula GalNAcα(Ser/Thr)ₘ and when n is 1, then the structure is sialyl-Tn antigen with formula SAα6GalNAcα(Ser/Thr)ₘ.
Ser/Thr indicates serine or threonine residues to which the small O-glycans are conjugated in the peptide sequence of protein. The variable m indicates when m is 1 that the epitope, which is recognized by the binder reagent includes in an embodiment at least part of the linking amino acid residue. The invention is further directed to antibodies recognizing at least part of peptide sequence close to the Ser/Thr-residue. In a preferred embodiment the binder has the same or similar specificity to the antibody clones used in examples.

Novel glycan binder reagent Fab1.4.24 The invention also revealed a novel glycan binder antibody reagent Fab1.4.24 (or Fab1) with several unusual oligosaccharide and sialic acid linkage structure binding specificites and high specificity in recognition of cryptic glycan epitopes.

It is further realized that the invention is in a preferred embodiment directed to lower molecular weight, and even more preferably monovalent, antibody derivatives such as single chain Fvs (scFvs), Fab fragments, Fab′ fragments, F(ab′) fragments, disulfide linked Fvs (sdFvs), Fvs, and fragments comprising or alternatively consisting of, either a VL or a VH domain. The low molecular weight antibody reagents including “single chain antibodies” are preferred because of the effective recognition of cryptic epitopes similarily as Fab1.4.24, partially because of high accessibility to membrane surfaces by lower molecular weight antibody reagents. These can be produced from other antibodies by routine proteolysis and/or recombinant expression technologies.

Ranges of presentation of cryptic epitopes. In a preferred embodiment the invention is directed to cryptic epitopes, which are revealed present as cryptic epitopes in a specific part of a cell population, represented as preferred lower and higher ranges:
a) lower range of binder positive labeled cells of the total cell population between about 1% and 50% of cells. The preferred conditions for lower range of observable binder positive cells include exposure to toxic, more preferably chelating agents such as EDTA. Preferred lower ranges includes especially over 20, even more preferably over 30 and most preferably over 40%, these are surprisingly high values especially in context of cell detachment, and more than generally observable for mesenchymal stem cells under normal detachment condition
b) higher range of binder positive labeled cells of the total cell population between about 51% and about 100% of cells. The preferred conditions for higher range observable binder positive cells include exposure to lipophilic agent or heat.
   The cells are preferably of a specific cell type, more preferably mesenchymal cells, even more preferably mesenchymal stem cells or differentiated mesenchymal cells. The invention is directed to novel cell populations produced by binding the novel i) binders such as 1.4.24 or ii) novel cell population produced by contacting the cell the binders, when the novel cells correspond to larger proportion of the cells than previously described and cells comprising membranes which present more effectively the novel cryptic epitopes, e.g. as shown by FACS diagrams in the Figures or cultivatable mesenchymal cells, the positively selected cells comprise binders (antibodies or reduced amount of antibodies, when part of antibodies are removed by inhibition with carbohydrates, and/or by protease), iii) cell population produced by contacting cells with the binders and isolating non-bound cell population, optionally further including cultivation of the non-bound cell population as a mesenchymal stem cell population comprising not substantial amount of the binder reagent.

Specific contexts for the use of the cell binding and analysis methods. The invention is further directed to the use of specific binder reagents, preferably antibodies against the cryptic epitope to assess membrane damage caused by the detachment method. In preferred embodiment the invention is directed to the evaluation of damage caused by cell detachment using a protease, preferably trypsin, and/or chelating agent during cell detachment or passaging process. In a preferred embodiment the quality control is directed to the verification of cell status with regard to harmful conditions potentially causing the exposure of the novel cryptic epitopes. In a preferred embodiment the cells are analyzed with regard to the cryptic epitopes in order to characterize a cell population.

Assay for the development or analysis of an antibody. The invention is further directed to the use of the target structures and specific glycan target structures for the screening of additional binders, preferably specific antibodies or lectins recognizing the terminal glycan structures and the use of the binders produced by the screening according to the invention. It is noted that for the screening there are various methods and steps described in the scientific literature.

Recognition of Structures From Glycome Materials and On Cell Surfaces By Binding Methods

The present invention revealed that beside the physiochemical analysis by mass spectrometry several methods are useful for the analysis of the structures. The preferred binders include:
i) Proteins such as antibodies, lectins and enzymes, ii) Peptides such as binding domains and sites of proteins, and synthetic library derived analogs such as phage display peptides, iii) Other polymers or organic scaffold molecules mimicking the peptide materials.
The peptides and proteins are preferably recombinant proteins or corresponding carbohydrate recognition domains derived thereof, when the proteins are selected from the group of monoclonal antibody, glycosidase, glycosyl transferring enzyme, plant lectin, animal lectin or a peptide mimetic thereof, and wherein the binder may include a detectable label structure.

The genus of enzymes in carbohydrate recognition is continuous to the genus of lectins (carbohydrate binding proteins without enzymatic activity). a) Native glycosyltransferases (Rauvala et al. (1983) PNAS (USA) 3991-3995) and glycosidases (Rauvala and Hakomori (1981) J. Cell Biol. 88, 149-159) have lectin activities. b) The carbohydrate binding enzymes can be modified to lectins by mutating the catalytic amino acid residues (see WO9842864; Aalto J. et al. Glycoconjugate J. (2001, 18(10); 751-8; Mega and Hase (1994) BBA 1200 (3) 331-3). c) Natural lectins, which are structurally homologous to glycosidases are also known, hence indicating the continuity of the genus enzymes and lectins (Sun, Y-J. et al. J. Biol. Chem. (2001) 276 (20) 17507-14). The genus of the antibodies as carbohydrate binding proteins without enzymatic activity is also very close to the concept of lectins, but antibodies are usually not classified as lectins.

Information on useful binder specificities including lectin and elongated antibody epitopes is available from reviews and monographs such as (Debray and Montreuil (1991) Adv. Lectin Res 4, 51-96; “The molecular immunology of complex carbohydrates” Adv Exp Med Biol (2001) 491 (ed Albert M Wu) Kluwer Academic/Plenum publishers, New York; “Lectins” second Edition (2003) (eds Sharon, Nathan and Lis, Halina) Kluwer Academic publishers Dordrecht, The Neatherlands and internet databases such as pubmed/espacenet or antibody databases such as www.glyco.is.ritsumei.ac.jp/epitope/, which list glycan specificities of monoclonal antibodies.

Antibodies. Known methods are used for the production of antibodies, e.g any suitable host animal is immunized, antibody is expressed from cloned immunoglobulin cDNAs and/or an antibody library such as phage display library is screened. Monoclonal antibody preparation include hybridoma techniques (Köhler et al., Nature, 256: 495-497, 1975; Kosbor et al., Immunology Today, 4: 72, 1983; Cole et al., Monoclonal Antibodies and Cancer Therapy, Alan R Liss, Inc., pp. 77-96, 1985), all incorporated herein by reference.

The invention is directed to the use of several reagents recognizing terminal epitopes together, preferably at least two reagents, more preferably at least three epitopes, even more preferably at least four, even more preferably at least five, even more preferably at least six, even more preferably at least seven, and most preferably at least 8 to recognize enough positive and negative targets together. The high specificity binders selectively and specifically recognizing elongated epitopes binds one of the elongated epitopes at least in the order of increasing preference, 5, 10, 20, 50, or 100 fold affinity. Methods for measuring the antibody binding affinities are well known in the art. The invention is also directed to the use of lower specificity antibodies capable of effective recognition of one elongated epitope but also at least one, preferably only one, additional elongated epitope with same terminal structure. The reagents are preferably used in arrays comprising in the order of increasing preference 5, 10, 20, 40 or 70 reagents including reagents shown in cell labeling experiments or variants thereof and optionally other binder reagents for detection of cell purity, differentiation, cell surface markers etc. e.g. ones described in PCT/FI2008/050019.

In a preferred embodiments the binder reagent, preferably an antibody, binds specifically or with higher activity/affinity to the target oligosaccharide sequence than other saccharide sequences, more preferably the specificity is practically exclusive allowing recognition of the target cryptic glycan oligosaccharide sequence but not other oligosaccharide sequences or at least not other non-cryptic glycan sequences by the specific detection method. In another preferred embodiment a lower specificity binder is used with a cell type which is verified to comprise not cross reactive structures for the lower specificity binder. The high specificity or exclusive binder is in a preferred embodiment an antibody recognizing at least in part a cryptic trisaccharide oligosaccharide sequence, more preferably at least part of tetrasaccharide oligosaccharide sequence and linkage structures between the residues. The verification may be performed with other binder reagents and/or by mass spectrometry e.g. as described in PCT/FI2008/050019.

Chemical definitions. Glycolipid and carbohydrate nomenclature is essentially according to recommendations by the IUPAC-IUB Commission on Biochemical Nomenclature (e.g. Carbohydrate Res. 1998, 312, 167; Carbohydrate Res. 1997, 297, 1; Eur. J. Biochem. 1998, 257, 29). It is assumed that Gal (galactose), Glc (glucose), GlcNAc (N-acetylglucosamine), GalNAc (N-acetylgalactosamine) and Neu5Ac are of the D-configuration, Fuc of the L-configuration, and all the monosaccharide units in the pyranose form. The amine group is as defined for natural galactose- and glucosamines on the 2-position of GalNAc or GlcNAc. Glycosidic linkages are shown partly in shorter and partly in longer nomenclature, the linkages of the sialic acid SA/Neu5X-residues α3 and α6 mean the same as α2-3 and α2-6, respectively, and with other monosaccharide residues α1-3, β1-3, β1-4, and β1-6 can be shortened as α3, β3, β4, and β6, respectively. Lactosamine refers to type II N-acetyllactosamine, Galβ4GlcNAc, and/or type I N-acetyllactosamine, Galβ3GlcNAc and sialic acid (SA) is N-acetylneuraminic acid (Neu5Ac) or N-glycolylneuraminic acid (Neu5Gc) or any other natural sialic acid including derivatives of Neu5X. The sialic acid are referred together as NeuNX or Neu5X, wherein preferably X is Ac or Gc. Occasionally Neu5Ac/Gc/X may be referred as NeuNAc/NeuNGc/NeuNX. Term “glycan” means here broadly oligosaccharide or polysaccharide chains present in human or animal glycoconjugates, especially on glycolipids or glycoproteins.

Glycan epitope or epitopes means oligosaccharide sequence and elongated epitope means reducing end elongated preferred oligosaccharide sequence variants.

“Oligosaccharide sequence” means specific sequence of glycosidically linked monosaccharide residues, including terminal and “core” sequences. In a preferred embodiment the or sialylated and fucosylated oligosaccharide sequences and/or oligosaccharide sequences except keratan sulfate are “terminal oligosaccharide sequence”. The core oligosaccharide sequences can be modified by non-reducing end monosaccharide residue(s). The expression “terminal oligosaccharide sequence” indicates that the oligosaccharide is not substituted to the non-reducing end terminal residue by another monosaccharide residue or residues. Preferably the non-reducing end of the oligosaccharide sequence consist of the oligosaccharide sequence and it is only modified from the reducing end of the oligosaccharide sequence, preferably it is glycosidically conjugated from the reducing end. Keratan sulfate typically comprises polysaccharide chain, which comprises keratan sulfate oligosaccharide chain epitopes such as sialylated non-reducing end terminal oligosaccharide chains.

“Saccharide” means monosaccharide or oligosaccharide epitope. The saccharide epitopes are preferably non-reducing end terminal saccharides, which may be elongated from reducing end. The elongating structure may be a natural sequence of the natural glycan recognized such as O-glycan, N-glycan or glycolipid (preferably glycospingolipid) structure. The monosaccharide residues are linked by alfa- or beta-glycosidic linkage to a non-monosaccharide material such as spacer structures linking glycans to polyacrylamides.

General method for isolation of cells or cellular components comprising the target structures. The invention is directed to a process of isolation of a fraction of cells or cell component involving contacting the epitope of binder molecule according to the invention. Corresponding target structures are expressed on stem cells and can be used to isolate the cell populations enriched with target structure.

The preferred method to isolate cellular component includes the following steps 1) Providing a cell sample, 2) Contacting the binder molecule according to the invention with the corresponding target structures on the cells or cell fractions. 3) Isolating the complex of the binder and target structure from at least part of the cells or cellular materials.

Preferred methods for isolation of cells include selection by immunomagnetic beads or by other cell sorting means, in a preferred embodiment by flow cytometry including FACS.

The isolation of cellular components according to the invention means production of a molecular fraction comprising increased or enriched amount of the glycans comprising the target structures according to the invention

In a preferred embodiment the invention is directed to the analysis cells, which have been exposed to the specific conditions causing necrosis. The analysis is in a preferred embodiment directed to the verification of possible exposure or its extent. “Exposed” means here exposed or one or several of the following status definitions of the cells: the cells i) may have been exposed to or ii) have actually been exposed to and/or iii) have been exposed to some extent or partially to and/or iv) need to be verified with regard to exposure to the specific conditions causing necrosis.

### EXAMPLES

### Example1

### Detection of Cells Damaged By EDTA Treatment By Anti-Glycan Antibodies

EDTA (Versene) is commonly used to detach cells from culture plates instead of trypsin, especially when there is the need to preserve cell surface proteins intact. In this example was is shown that 2 mM EDTA damages cell membranes so that the cells became propidium iodide permeable. Furthermore, it was shown that certain anti-glycan antibodies recognize epitopes that were accessible only in these damaged cells.

Materials and Methods Cell samples. Bone marrow (BM) derived mesenchymal stem cells (MSC:s). BM MSC:s were obtained as described by Leskelä et al. (2003). Briefly, bone marrow obtained during orthopaedic surgery was cultured in Minimun Essential Alpha-Medium supplemented with 20 mM HEPES, 10% fetal calf serum, penicillin-streptomycin and 2 mM L-glutamine (all from Gibco). After a cell attachment period of 2 days the cells were washed with PBS, subcultured further by plating the cells at a density of 2000-3000 cells/cm² in the same media and replacing the medium twice a week until near confluence. The cells used in the analyses were of passage 4-12.

Cord blood (CB) derived MSC:s Human term umbilical cord blood units were collected after delivery with informed consent of the mothers and the cord blood was processed within 24 hours of collection. Mononuclear cells (MNC:s) were isolated from each unit by Ficoll-Paque Plus (GE Healthcare Biosciences) density gradient centrifugation. The mononuclear cell fraction was plated on fibronectin (Sigma Aldrich)—coated 6-well plates (Nunc) at 10⁶ cells/well. Most of the non-adherent cells were removed as the medium was replaced the next day. The cells were cultured essentially as described for BM MSC:s above. The CB MSC:s used in the analyses were of passage 5-7.

Both BM and CB MSCs were analyzed by flow cytometry to be negative for CD14, CD34, CD45 and HLA-DR; and positive for CD13, CD29, CD44, CD90, CD105 and HLA-ABC. The cells were shown to be able to differentiate along osteogenic, adipogenic and chondrogenic lineages.

Osteogenic differentiation. Osteogenic differentiation of BM and CB MSC:s was induced by culturing the cells for 1-6 weeks in osteogenic induction medium: αMEM supplemented with 20 mM HEPES, 10% FCS, 2 mM glutamine, 0.1 μM dexamethasone, 10 mM γ-glycerophosphate, 0.05 mM ascorbic acid-2-phosphate, and penicillin-streptomycin.

Antibodies. Anti-Lewis c K21 was from Abcam (catalogue number ab3352); anti-blood group H type 2 B393 (DM3015), anti-Tn B1.1 (DM3218) and anti-sialyl Tn B35.1 (DM3197) were from Acris; anti-Lewis a PR5C5 and anti-sialyl Lewis a KM231were from Chemicon (CBL205 and MAB2095, respectively); anti-GT1b 3C96 was from US Biological (G2006-90A); anti-ganglioside antibody A2B5 was from Chemicon (MAB312R); Tra-1-81 and Tra-1-60 antibodies were from Chemicon (MAB4381 and MAB4361, respectively). In addition, a Fab fragment against a sialidase sensitive epitope (Fab1.4.24; Finnish Red Cross Blood Service) was used. Alexa Fluor 488 goat anti-mouse (Invitrogen) or FITC goat anti-human λ (Southern Biotech) was used as the secondary antibody. Propidium iodide (BD Biosciences) was used to assess membrane permeability.

Detachment of cells from culture plates. MSC:s were detached from culture plates by incubating with 2 mM EDTA-PBS (Versene) for 15 min at +37° C. or with 0.25% trypsin in 1 mM EDTA-PBS for 3 min at +37° C. Osteogenically differentiated MSC:s were detached by incubating with 10 mM EDTA-PBS (Versene) for 30 min (cells differentiated from cord blood MSC:s) or 1 h (cells differentiated from bone marrow MSC:s) at +37° C. or with 0.25% trypsin in 1 mM EDTA-PBS for 3 min at +37° C.

Flow cytometry. Cells (50 000) were incubated with primary antibodies (4 μl/100 μl diluted in 0.3% BSA-PBS) for 30 min at room temperature and washed once before incubating with secondary antibody (1:500) for 30 min at room temperature in the dark. Control cells were treated similarly but without primary antibody. When propidium iodide was used, it was added at 25 μl/50 000 cells and incubated for 15 min at room temperature after which the samples were placed on ice until analysis. Cells were analyzed with BD FACSAria (Becton Dickinson) using FITC detection at 488 nm or propidium iodide detection. Results were analyzed with BD FACSDiva software version 5.0.1 (Becton Dickinson).

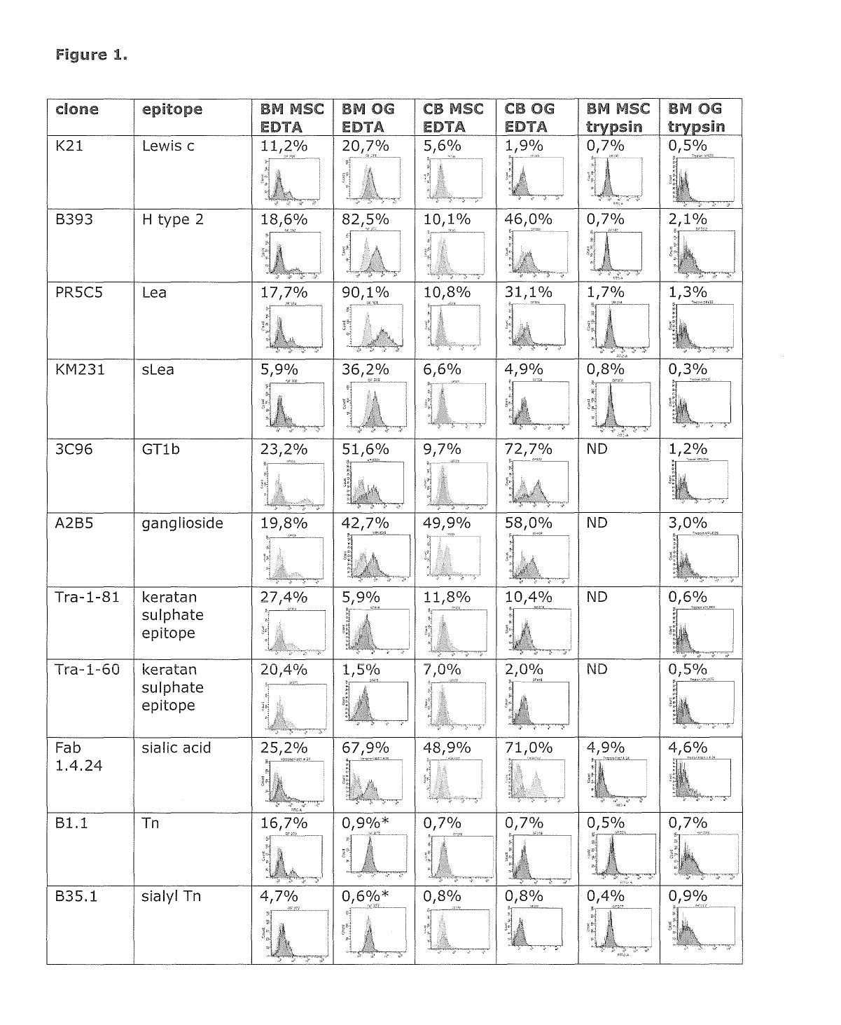

Results and Discussion A set of anti-glycan antibodies against blood group epitopes, gangliosides and keratan sulphate epitopes was shown to partially stain mesencymal stem cell populations (both bone marrow and cord blood derived) when the cells were detached from the culture plates with EDTA (FIG. 1). Generally around 20% of cells were stained, ranging from 5% to 45%. The number of positive cells was for the majority of antibodies higher in osteogenically differentiated cells, which were treated with a higher concentration of EDTA for a longer period of time, because they were more tightly attached to the substratum and more difficult to detach than undifferentiated mesenchymal stem cells. Fully differentiated osteoblasts (6 weeks of differentiation) did not stain with anti-Tn or anti-sialyl Tn, but cells at 3 and 4 weeks of osteogenic differentiation gave positive staining with anti-Tn and anti-sialyl Tn. When the cells were detached with trypsin, none of the antibodies recognized more than a few percent of either mesenchymal stem cells or osteogenically differentiated cells.

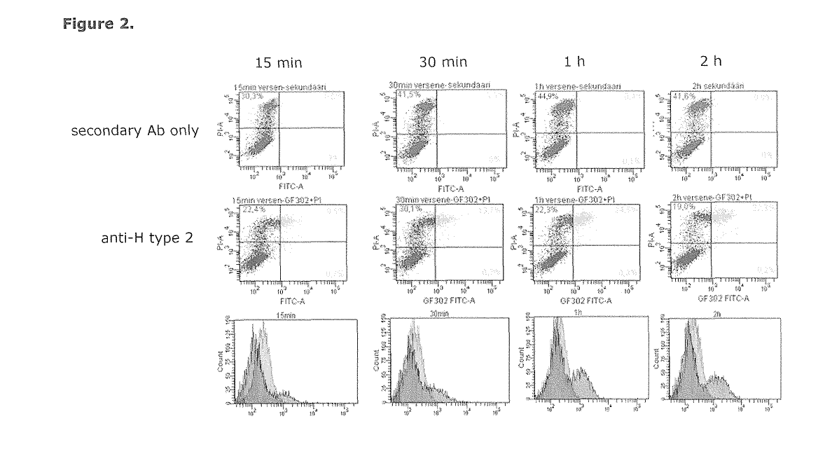

To further characterize the cells stained by the anti-glycan antibodies, the cells were double stained with anti-H type 2 blood group antigen and propidium iodide. Propidium iodide is a nucleic acid stain that is not permeable to the plasma membrane, and therefore does not enter intact cells. FIG. 2 shows that all cells that were stained with the anti-glycan antibody were also permeable to propidium iodide. Both the number of cells positive for the anti-glycan antibody staining and the number of propidium iodide positive cells increased when the duration of EDTA treatment was increased from 15 min to 2 hours.

Trypsin treatment may digest glycoprotein epitopes from the cell surface, which may cause staining with glycan antibodies to be positive in cells detached with EDTA and negative in cells detached with trypsin. However, in this example it seems more likely that the epitopes are intracellular and only became accessible when the cell membrane was damaged by the EDTA treatment, because only propidium iodide positive cells stained with the glycan antibodies. Furthermore, some of the antibodies used are against ganglioside epitopes, which are unlikely to be trypsin sensitive.

Detachment of cells with EDTA seems to cause membrane damage and reveal certain glycan epitopes. Antibodies against these epitopes may be used to assess membrane damage caused by the detachment method.

### Example2

### Detection of Necrotic and Permeabilized Cells By Anti-Glycan Antibodies

Cells were permeabilized by Triton X-100 or induced to undergo necrosis by heating at 56° C. for 30 min and analyzed by a set of glycan antibodies. Certain glycan antibodies were shown to bind to cells damaged by detachment methods, permeabilized cells and necrotic cells, and not to intact cells.

Materials and Methods Cell samples and antibodies were obtained as in Example 1; detachment of cells from culture plates and flow cytometry were carried out as in Example 1.

Induction of necrosis. Necrosis was induced by incubating the cells (detached with trypsin) in culture medium at +56° C. for 30 min.

Permeabilization of cells. Cells (detached with trypsin) were permeabilized by incubating them with 0.1% Triton X-100, 0.3% BSA, 2 mM EDTA-PBS for 10 min at room temperature.

Results and Discussion To extend the observation that membrane damage in EDTA treated cells exposes carbohydrate epitopes, mesenchymal stem cells were treated with 0.1% Triton X-100 to cause membrane permeabilization, or incubated at +56° C. for 30 min to induce necrosis. The cells were stained with propidium iodide, an anti-glycan Fab recognizing a sialidase-sensitive epitope, and anti-Lewis a.

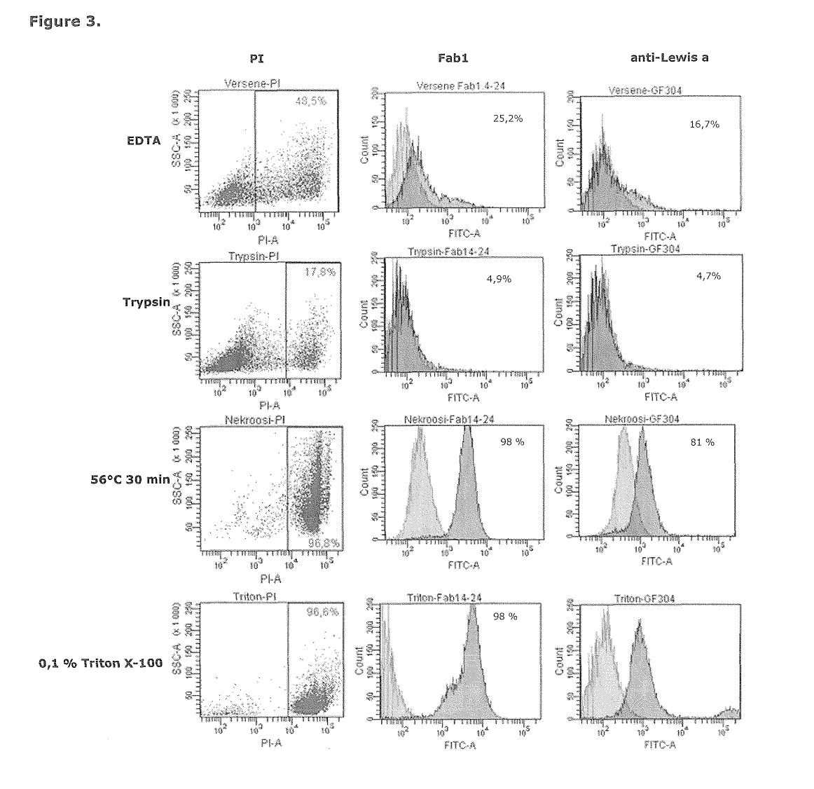

Detachment of cells with either EDTA or trypsin caused some membrane damage (48.5% and 17.5% of cells, respectively, became propidium iodide positive). The cells detached with EDTA or trypsin but otherwise untreated were weakly stained with the anti-glycan antibodies (FIG. 3).

Permeabilization and induction of necrosis resulted in all of the cells becoming propidium iodide positive. All or most of the cells became also positive for staining with both of the anti-glycan antibodies (FIG. 3).

The observation that the glycan epitopes become accessible in trypsin detached cells after permeabilization with Triton X-100 or induction of necrosis further supports the notion that the glycan epitopes become accessible in cells with membrane damage, rather than being digested with trypsin.

This example shows that certain glycan antibodies recognize epitopes that become accessible when the cell membrane is damaged by detergent permeabilization or induction of necrosis. These antibodies may be used to assess the extent of damage to cells caused by growth conditions or handling, for example detachment from culture plates or freezing and thawing.

### Example 3

### Proliferation of Mesenchymal Stem Cell Subpopulations Sorted on the Basis of Glycan Antibody Binding

Materials and methods Cells, cell culture, antibodies and FACS as described in Example 1.

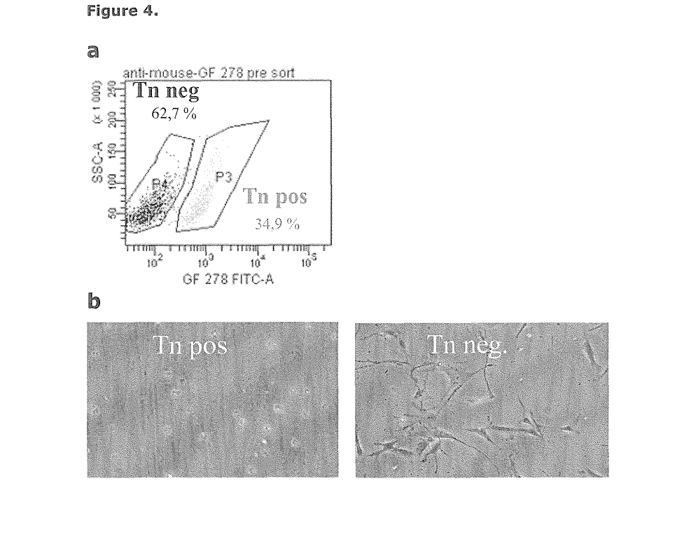

Results and Discussion Bone marrow mesenchymal stem cells were labeled with anti-Tn and sorted by FACS. Sorting is shown in FIG. 4a. The sorted subpopulations were plated separately at 1500 cells/cm² (14 000 cells per one well on a six well plate) in culture medium and let to attach and proliferate for three days. The Tn negative cells attached to the substratum and started proliferate, where as the Tn positive cells failed to attach or proliferate (FIG. 4b), suggesting that the Tn positive subpopulation does not consist of viable cells.

### Example 4

### Glycan Binding Specificity of Antibodies That Recognize a Cryptic Subpopulation of Mesenchymal Stem Cells

Materials and methods Antibodies: anti-GT1b 3C96 was from US Biological (catalogue number G2006-90A); Tra-1-81 and Tra-1-60 antibodies were from Chemicon (MAB4381 and MAB4361, respectively), anti-Tn B1.1 (DM3218) was from Acris. In addition, a Fab fragment against a sialidase sensitive epitope (Fab1.4.24; Finnish Red Cross Blood Service) was used.

Glycan microarray analysis was carried out by the Consortium for Functional Glycomics. Glycan microarrays were printed as described (Blixt 2004). Version 4.0 of the printed glycan array was used for analysis. Binding analysis was performed at 50 μg/ml of antibody. Data are reported as average RFU of 6 replicates after removal of highest and lowest values.

Results and discussion. The following anti-glycan antibodies recognize a cryptic/necrotic subpopulation of mesenchymal stem cells: anti-Lewis c (clone K21), anti-H type 2 (B393), anti-Lewis a (PR5C5), anti-sialyl Lewis a (KM231), anti-GT1b (3C96), anti-GQ1b (A2B5), anti-keratan sulfate (Tra-1-81; Tra-1-60), anti-Tn (B1.1), anti-sialyl Tn (B35.1), Fab-fragment Fab1.4.24. Of these, anti-GT1b (3C96), anti-keratan sulfate (Tra-1-81; Tra-1-60), anti-Tn (B1.1), and the Fab-fragment 1.4.24 were analyzed on a glycan microarray at the Consortium for Functional Glycomics. The glycan binding specificity anti-sialyl Lewis a (KM231) has been analyzed previously and is publicly available at the Consortium website (www.functionalglycomics.org). The best binding glycan structures for each antibody were analyzed and grouped according to structure type.

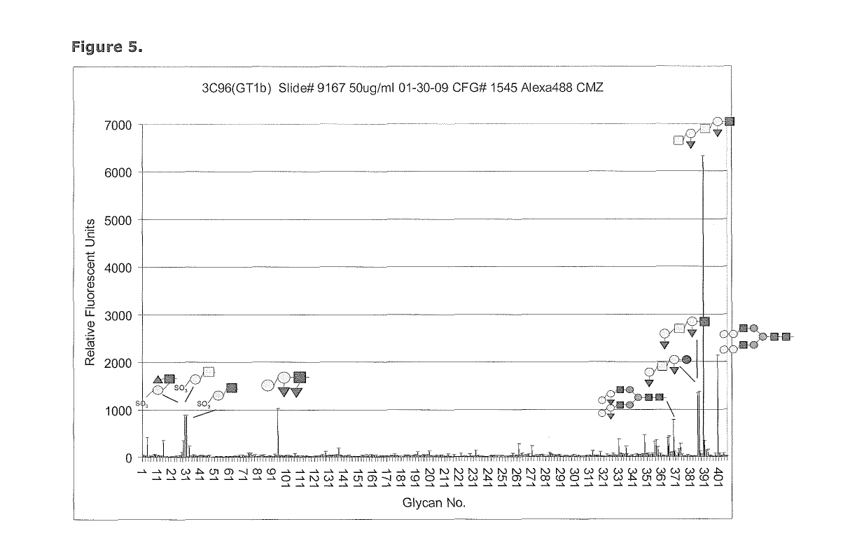

anti-GT1b. The glycan binding specificity of anti-GT1b 3C96 is shown in Table 1 and in FIG. 5. Unexpectedly, although the antibody is sold as and antibody against GT1b ganglioside, it did not bind to any ganglioside-like glycan structures on the glycan microarray. The best binding glycan structures for anti-GT1b were grouped into following structural categories: blood group antigens A, B and H, sulphated disaccharides, Lewis a, Lewis x, Lewis y, type 1 N-acetyllactosamine (Galβ1-3GlcNAc) and alpha-linked galactose.

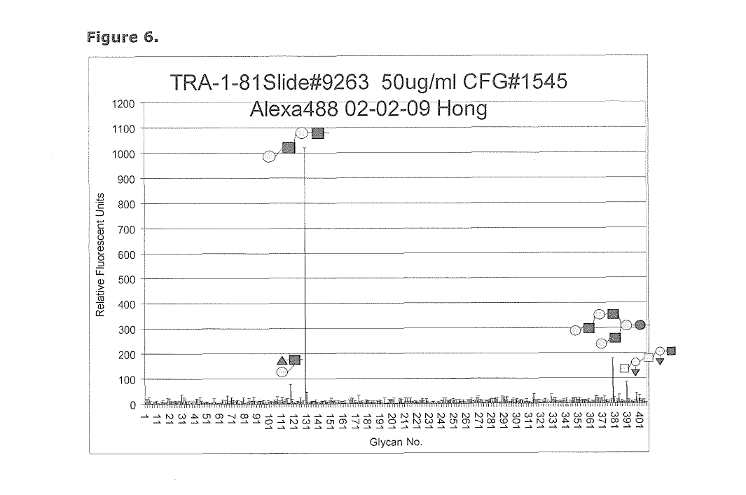

Tra-1-81. The glycan binding specificity of Tra-1-81 is shown in Table 2 and in FIG. 6. Tra-1-81 is reported to recognize a carbohydrate epitope on podocalyxin, possibly a keratan sulphate epitope (Schopperle and DeWolf (2007) Stem Cells 25(3) 723-730), however the glycans bound by Tra-1-81 have not been structurally characterized. On the glycan microarray Tra-1-81 bound to type 1 N-acetyllactosamine β1-3 linked to N-acetyllactosamine or lactose (Galβ1-3GlcNAcβ1-3Galβ1-4Glc(NAc). Tra-1-81 also weakly bound to Lewis a and blood group A1 antigen.

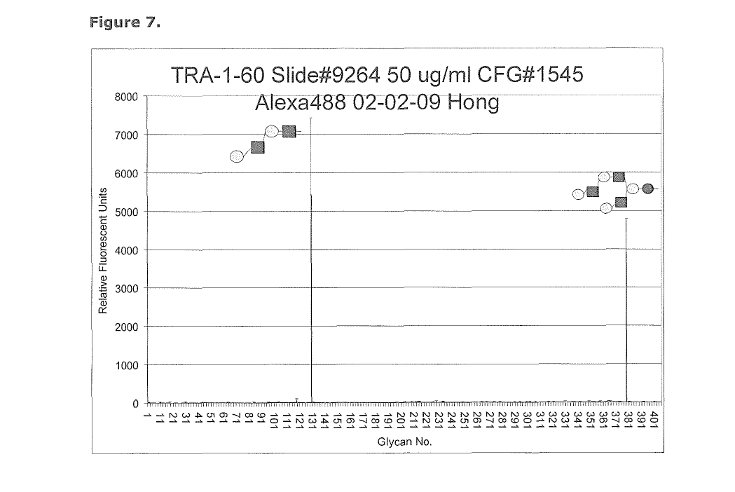

Tra-1-60 The glycan binding specificity of Tra-1-60 is shown in Table 3 and in FIG. 7. Tra-1-60 is reported to recognize a sialylated keratan sulphate epitope on podocalyxin (Schopperle and DeWolf (2007) Stem Cells 25(3) 723-730; Badcock et al. (1999) Cancer Res. 59, 4715-4719). On the glycan microarray Tra-1-60 exclusively bound to type 1 N-acetyllactosamine β1-3 linked to N-acetyllactosamine or lactose (Galβ1-3GlcNAcβ1-3Galβ1-4Glc(NAc). The discrepancy between the present results and the literature can be explained by the fact that both keratan sulphate and Galβ1-3GlcNAcβ1-3Galβ1-4Glc(NAc) are digested by the keratanase enzyme used in the analysis of the epitope by Badcock et al.

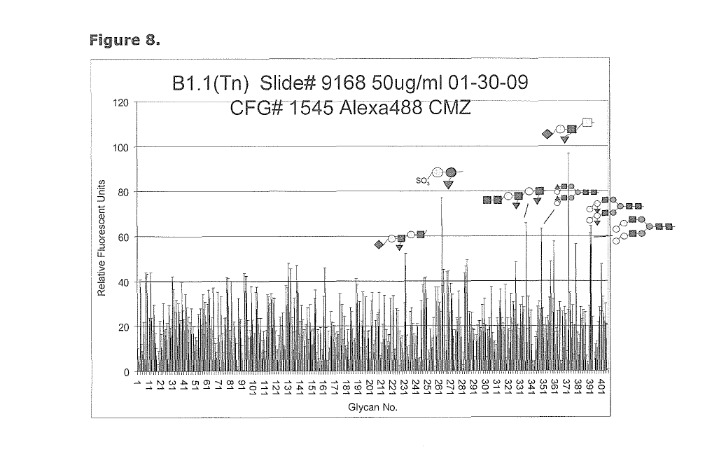

anti-Tn The glycan binding specificity of anti-Tn B1.1 is shown in Table 4 and in FIG. 8. Unexpectedly, the antibody did not bind to Tn-antigen (GalNAcα). The mAb B1.1 only showed very weak binding to any of the antigens on the glycan microarray. The best binding glycan structures for anti-Tn were grouped into following structural categories: type 1 N-acetyllactosamine, Lewis a, Lewis x, sialyl Lewis x, alpha-linked galactose and sulphated disaccharides.

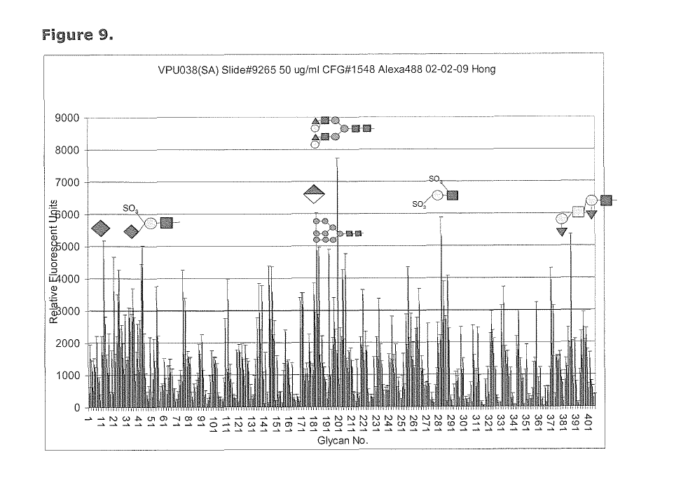

Fab 1.4.24 The glycan binding specificity of Fab1.4.24 (VPU038) is shown in Table 5 and in FIG. 9. The best binding glycan structures for Fab1.4.24 were grouped into following structural categories: Lewis a, Lewis x, sialyl Lewis x, type 2 polylactosamines, sulphated disaccharides, glucuronic acid, blood group antigens A2 and H, high mannose N-glycans, N-acetylneuraminic acid (especially α2,3-linked to type 2 N-acetyllactosamine), O-glycan core 1, kito-oligosaccharides.

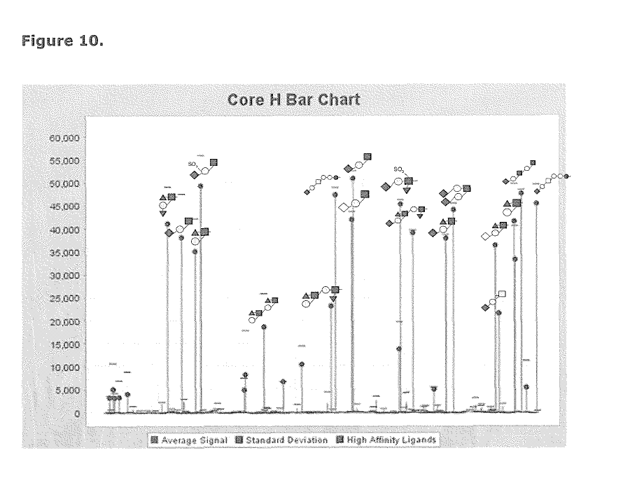

anti-sialyl Lewis a The glycan binding specificity of anti-sialyl Lewis a (KM231) has been analyzed previously and is publicly available at the Consortium for Functional Glycomics website (www.functionalglycomics.org). The data are presented here to allow comparison with the other anti-glycan antibodies that recognize a similar cryptic subpopulation of mesenchymal stem cells. The glycan binding specificity of anti-sialyl Lewis a KM231 is shown in Table 6 and in FIG. 10. In addition to sialyl Lewis a, mAb KM231 binds to Lewis a and α2,3-sialylated type 1 N-acetyllactosamine (Neu5Acα2-3Galβ1-3GlcNAc).

Common structural categories among the glycan epitopes defining a cryptic subpopulation of mesenchymal stem cells. The glycan structures bound by the anti-glycan antibodies that recognize a cryptic subpopulation of mesenchymal stem cells were grouped into structural categories. Table 7 shows structural categories bound by several of the antibodies. Lewis a (Galβ1-3(Fucα1-4)GlcNAc) was bound by anti-GT1b 3C96, anti-Tn B1-1, Tra-1-81, Fab 1.4.24 and anti-sLea KM231. Type 1 N-acetyl-lactosamine (Galβ1-3GlcNAc) was bound by anti-GT1b 3C96, anti-Tn B1-1, Tra-1-81 and Tra-1-60, and in an extended form (Galβ1-3GlcNAcβ1-3Galβ1-4Glc(NAc)) by anti-Tn B1-1, Tra-1-81 and Tra-1-60. Sulphated disaccharides and sialyl Lewis x (Neu5Acα2-3Galβ1-4(Fucα1-3)GlcNAc) were bound by anti-GT1b 3C96, anti-Tn B1-1, Fab 1.4.24 and anti-sLea KM231. Blood group A antigens (GalNAcα1-3(Fucα1-2)Galβ1-3GalNAcα1-3(Fucα1-2)Galβ1-4Glc(NAc) or Fucα1-2Galβ1-3GalNAcα1-3(Fucα1-2)Galβ1-4Glc(NAc) were bound by anti-GT1b 3C96, Tra-1-81 and Fab 1.4.24. Lewis x (Galβ1-4(Fucα1-3)GlcNAc) was bound by anti-Tn B1-1, Tra-1-81 and Tra-1-60. Some of the anti-glycan antibodies binding to a similar cryptic subpopulation of mesenchymal stem cells, whose specificity was not analyzed in the present experiments, are reported to bind to some of the same categories: anti-Lewis c (K21) to type 1 N-acetyllactos-amines and anti-Lewis a (PR5C5) to Lewis a. Since the antibodies that recognize a cryptic subpopulation of mesenchymal stem cells cross react with certain structural categories, especially type 1 antigens, the staining of these epitopes, possibly intracellularly, may form the structural basis of the subpopulation.

TABLE 1
Glycan binding specificity of mAb 3C96 (anti-GT1b).

| Chart # | Masterlist Name | RFU | STDEV |
|---|---|---|---|
| 390 | GalNAca1-3(Fuca1-2)Galb1-SGalNAca1-3(Fuca1-2)Galb1-4GlcNAcb-Sp0 | 6196 | 265 |
| 386 | Fuca1-2Galb1-3GalNAca1-3(Fuca1-2)Galb1-4Glcb-Sp0 | 1290 | 104 |
| 387 | Fuca1-2Galb1-3GalNAca1-3(Fuca1-2)Galb1-4GlcNAcb-Sp0 | 1264 | 232 |
| 400 | Gala1-4Galb1-3GlcNacb1-2Mana1-3(Gala1-4Galb1-3GlcNacb1-2Mana1-6)Manb1-4GlcNacb1-4GlcNa [symbol] | 1246 | 1781 |
| 31 | [3OSO3]Galb1-3GlcNAcb-Sp8 | 872 | 36 |
| 30 | [3OSO3]Galb1-3GalNAca-Sp8 | 848 | 97 |
| 95 | Gala1-3(Fuca1-2)Galb1-4(Fuca1-3)GlcNAcb-Sp0 | 550 | 948 |
| 369 | Gala1-3(Fuca1-2)Galb1-3GlcNAcb1-2Mana1-3(Gala1-3(Fuca1-2)Galb1-3GlcNAcb1-2Mana1-6)Manb1 [symbol] | 407 | 742 |
| 29 | [3OSO3]Galb1-3(Fuca1-4)GlcNAcb-Sp8 | 348 | 19 |
| 15 | Galb-Sp8 | 300 | 119 |
| 349 | Galb1-3(Fuca1-4)GlcNAcb1-2Mana1-3(Galb1-3(Fuca1-4)GlcNAcb1-2Mana1-6)Manb1-4GlcNAcb1-4G [symbol] | 299 | 343 |
| 366 | Gala1-3(Fuca1-2)Galb1-4GlcNAcb1-2Mana1-3(Gala1-3(Fuca1-2)Galb1-4GlcNAcb1-2Mana1-6)Manb1- [symbol] | 274 | 323 |
| 4 | Galb1-3GlcNAcb1-2Mana1-3(Galb1-3GlcNAcb1-2Mana1-6)Manb1-4GlcNAcb1-4GlcNAcb-Sp19 | 269 | 314 |
| 391 | Gala1-3Galb1-3GlcNAcb1-2Mana1-3(Gala1-3Galb1-3GlcNAcb1-2Mana1-6)Manb1-4GlcNAcb1-4GlcN [symbol] | 246 | 196 |
| 356 | Fuca1-2Galb1-4GlcNAcb1-2Mana1-3(Fuca1-2Galb1-4GlcNAcb1-2Mana1-6)Manb1-4GlcNAcb1-4GlcN [symbol] | 241 | 198 |
| 365 | GalNAca1-3(Fuca1-2)Galb1-4GlcNAcb1-2Mana1-3(GalNAca1-3(Fuca1-2)Galb1-4GlcNAcb1-2Mana1-6 [symbol] | 229 | 342 |
| 357 | Fuca1-2Galb1-4(Fuca1-3)GlcNAcb1-2Mana1-3(Fuca1-2Galb1-4(Fuca1-3)GlcNAcb1-2Mana1-6)Manb1 [symbol] | 225 | 255 |
| 331 | (Neu5Aca2-3-Galb1-3)(((Neu5Aca2-3-Galb1-4(Fuca1-3))GlcNAcb1-6)GalNAc-Sp14 | 219 | 317 |
| 33 | [3OSO3]Galb1-4[6OSO3]GlcNAcb-Sp8 | 203 | 66 |
| 374 | GalNAcb1-4GlcNAcb1-2Mana1-6(GalNAcb1-4GlcNAcb1-2Mana1-6)Manb1-4GlcNAcb1-4GlcNAc-Sp12 [symbol] | 200 | 149 |
| 336 | GlcNAca1-4Galb1-4GlcNAcb1-3Galb1-4(Fuca1-3)GlcNAcb1-3Galb1-4(Fuca1-3)GlcNAcb-Sp0 | 195 | 65 |
| 137 | Galb1-4(Fuca1-3)GlcNAcb1-4Galb1-4(Fuca1-3)GlcNAcb1-4Galb1-4(Fuca1-3)GlcNAcb-Sp0 | 169 | 35 |
| 262 | [3OSO3]Galb1-4(Fuca1-3)[6OSO3]Glc-Sp0 | 164 | 224 |
| 358 | Gala1-3Galb1-4GlcNAcb1-2Mana1-3(Gala1-3Galb1-4GlcNAcb1-2Mana1-6)Manb1-4GlcNAcb1-4GlcN [symbol] | 162 | 91 |
| 271 | Galb1-3(Fuca1-4)GlcNAcb1-3Galb1-3(Fuca1-4)GlcNAcb-Sp0 | 139 | 185 |
| 373 | Neu5Aca2-3Galb1-4(Fuca1-3)GlcNAcb1-3GalNAca-Sp14 | 136 | 60 |
| 392 | Gala1-3Galb1-3(Fuca1-4)GlcNAcb1-2Mana1-3(Gala1-3Galb1-3(Fuca1-4)GlcNAcb1-2Mana1-6)Manb1 [symbol] | 124 | 25 |
| 313 | Neu5Aca2-3Galb1-3(Neu5Aca2-6)GalNAca-Sp14 | 117 | 34 |
| 232 | Neu5Aca2-3Galb1-4(Fuca1-3)GlcNAcb1-3Galb1-4GlcNAcb-Sp8 | 117 | 63 |
| 200 | Fuca1-3(Galb1-4)GlcNAcb1-2Mana1-3(Fuca1-3(Galb1-4)GlcNAcb1-2Mana1-6)Manb1-4GlcNAcb1-4G [symbol] | 103 | 62 |
| 393 | Neu5Aca2-3Galb1-3GlcNAcb1-2Mana1-3(Neu5Aca2-3Galb1-3GlcNAcb1-2Mana1-6)Manb1-4GlcNAcb [symbol] | 90 | 128 |
| 28 | [3OSO3]Galb1-4[6OSO3]Glcb-Sp8 | 90 | 34 |
| 128 | Galb1-SGalNAcb1-4Galb1-4Glcb-Sp8 | 90 | 73 |
| 370 | Fuca1-2Galb1-3(Fuca1-4)GlcNAcb1-2Mana1-3(Fuca1-2Galb1-3(Fuca1-4)GlcNAcb1-2Mana1-6)Manb1 [symbol] | 89 | 39 |
| 283 | [3OSO3]Galb1-4[6OSO3]GlcNAcb-Sp0 | 87 | 27 |
| 74 | Fuca1-2Galb-Sp8 | 86 | 19 |
| 388 | Galb1-3GlcNAcb1-3GalNAca-Sp14 | 82 | 72 |
| 76 | Fuca1-4GlcNAcb-Sp8 | 80 | 22 |
| 192 | Mana1-2Mana1-6(Mana1-3)Mana1-6(Mana1-2Mana1-2Mana1-3)Manb1-4GlcNAcb1-4GlcNAcb-Sp12 | 79 | 74 |
| 368 | GalNAca1-3(Fuca1-2)Galb1-3GlcNAcb1-2Mana1-3(GalNAca1-3(Fuca1-2)Galb1-3GlcNAcb1-2Mana1- [symbol] | 74 | 52 |
| 401 | Gala1-4Galb1-4GlcNacb1-2Mana1-3(Gala1-4Galb1-4GlcNacb1-2Mana1-6)Manb1-4GlcNacb1-4GlcNa [symbol] | 74 | 9 |
| 318 | Neu5Aca2-6Galb1-4GlcNAcb1-2Mana1-3(Neu5Aca2-6Galb1-4GlcNAcb1-2Mana1-6)Manb1-4GlcNAcb [symbol] | 74 | 70 |
| 222 | Neu5Aca2-3Galb1-3GalNAcb1-3Gala1-4Galb1-4Glcb-Sp0 | 73 | 16 |
| 367 | Gala1-3Galb1-4(Fuca1-3)GlcNAcb1-2Mana1-3(Gala1-3Galb1-4(Fuca1-3)GlcNAcb1-2Mana1-6)Manb1 [symbol] | 72 | 40 |
| 75 | Fuca1-3GlcNAcb-Sp8 | 68 | 3 |
| 107 | Gala1-4(Fuca1-2)Galb1-4GlcNAcb-Sp8 | 68 | 19 |
| 264 | [3OSO3]Galb1-4(Fuca1-3)[6OSO3]GlcNAc-Sp8 | 65 | 18 |
| 350 | [6OSO3]GlcNAcb1-3Galb1-4GlcNAc-b-Sp0 | 64 | 15 |
| 348 | Galb1-3GlcNAcb1-2Mana1-3(Galb1-3GlcNAcb1-2Mana1-6)Manb1-4GlcNAcb1-4(Fuca1-6)GlcNacb-Sp [symbol] | 64 | 14 |
| 284 | [3OSO3][4OSO3]Galb1-4GlcNAcb-Sp0 | 63 | 17 |

50 best binders out of the 442 glycan structures on the microarray are shown. [symbol]
indicates data missing or illegible when filed

TABLE 2
Glycan binding specificity of Tra-1-81.

| Chart # | Masterlist Name | RFU | STDEV |
|---|---|---|---|
| 130 | Galb1-3GlcNAcb1-3Galb1-4GlcNAcb-Sp0 | 721 | 597 |
| 379 | Galb1-3GlcNacb1-3(Galb1-3GlcNacb1-3Galb1-4GlcNacb1-6)Galb1-4Glcb-Sp0 | 147 | 64 |
| 390 | GalNAca1-3(Fuca1-2)Galb1-3GalNAca1-3(Fuca1-2)Galb1-4GlcNAcb-Sp0 | 72 | 26 |
| 118 | Galb1-3(Fuca1-4)GlcNAcb-Sp8 | 53 | 51 |
| 131 | Galb1-3GlcNAcb1-3Galb1-4Glcb-Sp10 | 34 | 24 |
| 30 | [3OSO3]Galb1-3GalNAca-Sp8 | 30 | 15 |
| 329 | GalNAca1-3(Fuca1-2)Galb1-4GlcNAcb1-3Galb1-4GlcNAcb-Sp0 | 26 | 19 |
| 365 | GalNAca1-3(Fuca1-2)Galb1-4GlcNAcb1-2Mana1-3(GalNAca1-3(Fuca1-2)Galb1-4GlcNAcb1-2Mana1- [symbol] | 25 | 13 |
| 315 | Neu5Aca2-3Galb1-4GlcNAcb1-2Mana1-3(Neu5Aca2-6Galb1-4GlcNAcb1-2Mana1-6)Manb1-4GlcNAcb [symbol] | 25 | 25 |
| 398 | Galb1-4(Fuca1-3)GlcNacb1-3GalNaca-Sp14 | 24 | 33 |
| 289 | Galb1-3GalNAca-Sp16 | 24 | 10 |
| 70 | Fuca1-2Galb1-4GlcNAcb1-3Galb1-4GlcNAcb1-3Galb1-4GlcNAcb-Sp0 | 23 | 5 |
| 270 | Fuca1-2Galb1-4[6OSO3]Glc-Sp0 | 23 | 13 |
| 366 | Gala1-3(Fuca1-2)Galb1-4GlcNAcb1-2Mana1-3(Gala1-3(Fuca1-2)Galb1-4GlcNAcb1-2Mana1-6)Manb1 [symbol] | 22 | 13 |
| 88 | GalNAcb1-3(Fuca1-2)Galb-Sp8 | 21 | 16 |
| 173 | GlcNAcb1-6(Galb1-3)GalNAca-Sp8 | 21 | 25 |
| 349 | Galb1-3(Fuca1-4)GlcNAcb1-2Mana1-3(Galb1-3(Fuca1-4)GlcNAcb1-2Mana1-6)Manb1-4GlcNAcb1-4Gl [symbol] | 21 | 4 |
| 370 | Fuca1-2Galb1-3(Fuca1-4)GlcNAcb1-2Mana1-3(Fuca1-2Galb1-3(Fuca1-4)GlcNAcb1-2Mana1-6)Manb1 [symbol] | 20 | 8 |
| 80 | GalNAca1-3(Fuca1-2)Galb1-4GlcNAcb-Sp0 | 20 | 11 |
| 67 | Fuca1-2Galb1-4(Fuca1-3)GlcNAcb-Sp0 | 20 | 24 |
| 384 | Galb1-4GlcNacb1-2(Galb1-4GlcNacb1-4)Mana1-3(Galb1-4GlcNacb1-2(Galb1-4GlcNacb1-6)Mana1-6) [symbol] | 20 | 27 |
| 215 | Neu5Aca2-3Galb1-3[6OSO3]GlcNAc-Sp8 | 20 | 3 |
| 45 | [6OSO3]GlcNAcb-Sp8 | 20 | 18 |
| 4 | Galb1-3GlcNAcb1-2Mana1-3(Galb1-3GlcNAcb1-2Mana1-6)Manb1-4GlcNAcb1-4GlcNAcb-Sp19 | 19 | 16 |
| 111 | Gala1-4GlcNAcb-Sp8 | 19 | 16 |
| 290 | Galb1-3(Neu5Aca2-3Galb1-4(Fuca1-3)GlcNAcb1-6)GalNAca-Sp14 | 19 | 11 |
| 225 | Neu5Aca2-3Galb1-3GlcNAcb-Sp8 | 19 | 2 |
| 284 | [3OSO3][4OSO3]Galb1-4GlcNAcb-Sp0 | 19 | 9 |
| 107 | Gala1-4(Fuca1-2)Galb1-4GlcNAcb-Sp8 | 18 | 21 |
| 126 | Galb1-3GalNAcb1-3Gala1-4Galb1-4Glcb-Sp0 | 18 | 12 |
| 202 | Neu5Aca2-8Neu5Aca2-8Neu5Aca2-8Neu5Aca2-3(GalNAcb1-4)Galb1-4Glcb-Sp0 | 18 | 11 |
| 400 | Gala1-4Galb1-3GlcNacb1-2Mana1-3(Gala1-4Galb1-3GlcNacb1-2Mana1-6)Manb1-4GlcNacb1-4GlcNa [symbol] | 18 | 27 |
| 42 | [6OSO3]Galb1-4GlcNAcb-Sp8 | 18 | 7 |
| 168 | GlcNAcb1-4(GlcNAcb1-6)GalNAca-Sp8 | 18 | 8 |
| 2 | Neu5Aca2-8Neu5Aca2-8Neu5Acb-Sp8 | 18 | 2 |
| 285 | [6OSO3]Galb1-4[6OSO3]GlcNAcb-Sp0 | 18 | 11 |
| 115 | Galb1-3(Fuca1-4)GlcNAcb1-3Galb1-4GlcNAcb-Sp0 | 17 | 8 |
| 314 | Neu5Aca2-3Galb1-3GalNAca-Sp14 | 17 | 9 |
| 194 | Mana1-3(Mana1-6)Mana-Sp9 | 17 | 11 |
| 56 | Fuca1-2Galb1-SGalNAcb1-3Gala1-4Galb1-4Glcb-Sp9 | 17 | 8 |
| 352 | KDNa2-6Galb1-4GlcNAc-Sp0 | 17 | 10 |
| 84 | GalNAca1-3GalNAcb-Sp8 | 17 | 13 |
| 244 | Neu5Aca2-6Galb1-4GlcNAcb-Sp8 | 17 | 10 |
| 324 | Neu5Aca2-6Galb1-4GlcNAcb1-3Galb1-3GlcNAcb-Sp0 | 17 | 4 |
| 167 | GlcNAcb1-4-MDPLys | 16 | 15 |
| 228 | Neu5Aca2-3Galb1-4(Fuca1-3)GlcNAcb1-3Galb1-4(Fuca1-3)GlcNAcb1-3Galb1-4(Fuca1-3)GlcNAcb-Sp [symbol] | 16 | 7 |
| 151 | Galb1-4GlcNAcb-Sp0 | 16 | 12 |
| 32 | [3OSO3]Galb1-4(Fuca1-3)GlcNAcb-Sp8 | 16 | 17 |
| 372 | NeuAca2-6Galb1-4GlcNAcb1-3GalNAc-Sp14 | 16 | 27 |
| 198 | Neu5Aca2-6Galb1-4GlcNAcb1-2Mana1-3(Neu5Aca2-3Galb1-4GlcNAcb1-2Mana1-6)Manb1-4GlcNAcb [symbol] | 16 | 6 |

50 glycan structures giving the highest signals out of the 442 glycan structures on the microarray are shown. [symbol]
indicates data missing or illegible when filed

TABLE 3
Glycan binding specificity of Tra-1-60.

| Chart # | Masterlist Name | RFU | STDEV |
|---|---|---|---|
| 130 | Galb1-SGlcNAcb1-3Galb1-4GlcNAcb-Sp0 | 5435 | 3990 |
| 379 | Galb1-SGlcNacb1-3(Galb1-3GlcNacb1-3Galb1-4GlcNacb1-6)Galb1-4Glcb-Sp0 | 3910 | 1754 |
| 118 | Galb1-3(Fuca1-4)GlcN Acb-Sp8 | 57 | 95 |
| 228 | Neu5Aca2-3Galb1-4(Fuca1-3)GlcNAcb1-3Galb1-4(Fuca1-3)GlcNAcb1-3Galb1-4(Fuca1-3)GlcNAcb-Sp [symbol] | 38 | 12 |
| 349 | Galb1-3(Fuca1-4)GlcNAcb1-2Mana1-3(Galb1-3(Fuca1-4)GlcNAcb1-2Mana1-6)Manb1-4GlcNAcb1-4G [symbol] | 33 | 15 |
| 30 | [3OSO3]Galb1-3GalNAca-Sp8 | 32 | 20 |
| 366 | Gala1-3(Fuca1-2)Galb1-4GlcNAcb1-2Mana1-3(Gala1-3(Fuca1-2)Galb1-4GlcNAcb1-2Mana1-6)Manb1 [symbol] | 29 | 21 |
| 365 | GalNAca1-3(Fuca1-2)Galb1-4GlcNAcb1-2Mana1-3(GalNAca1-3(Fuca1-2)Galb1-4GlcNAcb1-2Mana1- [symbol] | 27 | 11 |
| 18 | GalNAcb-Sp8 | 27 | 10 |
| 374 | GalNAcb1-4GlcNAcb1-2Mana1-6(GalNAcb1-4GlcNAcb1-2Mana1-6)Manb1-4GlcNAcb1-4GlcNAc-Sp1 [symbol] | 24 | 5 |
| 363 | Fuca1-2Galb1-3GlcNAcb1-3(Galb1-4(Fuca1-3)GlcNAcb1-6)Galb1-4Glc-Sp21 | 22 | 6 |
| 336 | GlcNAca1-4Galb1-4GlcNAcb1-3Galb1-4(Fuca1-3)GlcNAcb1-3Galb1-4(Fuca1-3)GlcNAcb-Sp0 | 22 | 16 |
| 381 | Fuca1-2Galb1-3(Fuca1-4)GlcNAcb1-3(Galb1-4GlcNAcb1-6)Galb1-4Glc-Sp21 | 21 | 9 |
| 214 | Neu5Aca2-3GalNAcb1-4GlcNAcb-Sp0 | 21 | 18 |
| 392 | Gala1-3Galb1-3(Fuca1-4)GlcNAcb1-2Mana1-3(Gala1-3Galb1-3(Fuca1-4)GlcNAcb1-2Mana1-6)Manb1 [symbol] | 20 | 11 |
| 317 | Neu5Aca2-6Galb1-4GlcNAcb1-2Mana1-3(GlcNAcb1-2Mana1-6)Manb1-4GlcNAcb1-4GlcNAcb-Sp12 | 20 | 5 |
| 43 | [6OSO3]Galb1-4[6OSO3]Glcb-Sp8 | 20 | 17 |
| 358 | Gala1-3Galb1-4GlcNAcb1-2Mana1-3(Gala1-3Galb1-4GlcNAcb1-2Mana1-6)Manb1-4GlcNAcb1-4GlcN [symbol] | 20 | 21 |
| 233 | Neu5Aca2-3Galb1-4GlcNAcb1-3Galb1-4(Fuca1-3)GlcNAc-Sp0 | 20 | 24 |
| 226 | Neu5Aca2-3Galb1-4[6OSO3]GlcNAcb-Sp8 | 20 | 6 |
| 373 | Neu5Aca2-3Galb1-4(Fuca1-3)GlcNAcb1-3GalNAca-Sp14 | 20 | 7 |
| 274 | Galb1-3(Neu5Aca2-3Galb1-4GlcNacb1-6)GalNAca-Sp14 | 20 | 12 |
| 203 | Neu5Aca2-8Neu5Aca2-8Neu5Aca2-3(GalNAcb1-4)Galb1-4Glcb-Sp0 | 19 | 16 |
| 104 | Gala1-3Galb1-4GlcN Acb-Sp8 | 18 | 12 |
| 337 | GlcNAca1-4Galb1-4GlcNAcb1-3Galb1-4GlcNAcb-Sp0 | 18 | 10 |
| 330 | GalNAca1-3(Fuca1-2)Galb1-4GlcNAcb1-3Galb1-4GlcNAcb1-3Galb1-4GlcNAcb-Sp0 | 17 | 26 |
| 215 | Neu5Aca2-3Galb1-3[6OSO3]GlcNAc-Sp8 | 17 | 12 |
| 131 | Galb1-3GlcNAcb1-3Galb1-4Glcb-Sp10 | 17 | 11 |
| 352 | KDNa2-6Galb1-4GlcNAc-Sp0 | 17 | 4 |
| 342 | Neu5Aca2-6Galb1-4GlcNAcb1-2Mana1-3Manb1-4GlcNAcb1-4GlcNAc-Sp12 | 17 | 15 |
| 290 | Galb1-3(Neu5Aca2-3Galb1-4(Fuca1-3)GlcNAcb1-6)GalNAca-Sp14 | 17 | 10 |
| 249 | Neu5Aca2-6Galb-Sp8 | 16 | 15 |
| 209 | Neu5Aca2-3(GalNAcb1-4)Galb1-4GlcNAcb-Sp8 | 16 | 12 |
| 10 | Fuca-Sp9 | 16 | 13 |
| 300 | GlcAb1-3GlcNAcb-Sp8 | 15 | 14 |
| 278 | Galb1-4(Fuca1-3)[6OSO3]Glc-Sp0 | 15 | 13 |
| 353 | KDNa2-3Galb1-4Glc-Sp0 | 15 | 9 |
| 64 | Fuca1-2Galb1-3GlcNAcb-Sp8 | 15 | 8 |
| 302 | GlcNAcb1-2Mana1-3(GlcNAcb1-2Mana1-6)Manb1-4GlcNAcb1-4GlcNAcb-Sp12 | 15 | 11 |
| 1 | Neu5Aca2-8Neu5Acb-Sp17 | 15 | 18 |
| 401 | Gala1-4Galb1-4GlcNacb1-2Mana1-3(Gala1-4Galb1-4GlcNacb1-2Mana1-6)Manb1-4GlcNacb1-4GlcNa [symbol] | 15 | 10 |
| 264 | [3OSO3]Galb1-4(Fuca1-3)[6OSO3]GlcNAc-Sp8 | 14 | 8 |
| 362 | Neu5Aca2-6GlcNAcb1-4GlcNAcb1-4GlcNAc-Sp21 | 14 | 14 |
| 384 | Galb1-4GlcNacb1-2(Galb1-4GlcNacb1-4)Mana1-3(Galb1-4GlcNacb1-2(Galb1-4GlcNacb1-6)Mana1-6) [symbol] | 14 | 3 |
| 173 | GlcNAcb1-6(Galb1-3)GalNAca-Sp8 | 14 | 4 |
| 162 | GlcNAcb1-3Galb1-3GalNAca-Sp8 | 14 | 5 |
| 96 | Gala1-3(Fuca1-2)Galb1-4GlcNAc-Sp0 | 14 | 21 |
| 152 | Galb1-4GlcNAcb-Sp8 | 14 | 8 |
| 48 | Mana1-3(Mana1-6)Manb1-4GlcNAcb1-4GlcNAcb-Sp13 | 14 | 8 |
| 268 | Fuca1-2[6OSO3]Galb1-4[6OSO3]Glc-Sp0 | 14 | 10 |

50 glycan structures giving the highest signals out of the 442 glycan structures on the microarray are shown. [symbol]
indicates data missing or illegible when filed

TABLE 4
Glycan binding specificity of anti-Tn (B1.1).

| Chart # | Masterlist Name | RFU | STDEV |
|---|---|---|---|
| 373 | Neu5Aca2-3Galb1-4(Fuca1-3)GlcNAcb1-3GalNAca-Sp14 | 81 | 31 |
| 263 | [3OSO3]Galb1-4(Fuca1-3)Glc-Sp0 | 68 | 18 |
| 336 | GlcNAca1-4Galb1-4GlcNAcb1-3Galb1-4(Fuca1-3)GlcNAcb1-3Galb1-4(Fuca1-3)GlcNAcb-Sp0 | 62 | 8 |
| 349 | Galb1-3(Fuca1-4)GlcNAcb1-2Mana1-3(Galb1-3(Fuca1-4)GlcNAcb1-2Mana1-6)Manb1-4GlcNAcb1-4G [symbol] | 57 | 13 |
| 392 | Gala1-3Galb1-3(Fuca1-4)GlcNAcb1-2Mana1-3(Gala1-3Galb1-3(Fuca1-4)GlcNAcb1-2Mana1-6)Manb1 [symbol] | 56 | 17 |
| 391 | Gala1-3Galb1-3GlcNAcb1-2Mana1-3(Gala1-3Galb1-3GlcNAcb1-2Mana1-6)Manb1-4GlcNAcb1-4GlcN [symbol] | 56 | 11 |
| 360 | Galb1-3(Fuca1-4)GlcNAcb1-2Mana1-3(Galb1-3(Fuca1-4)GlcNAcb1-2Mana1-6)Manb1-4GlcNAcb1-4(F [symbol] | 52 | 10 |
| 232 | Neu5Aca2-3Galb1-4(Fuca1-3)GlcNAcb1-3Galb1-4GlcNAcb-Sp8 | 47 | 11 |
| 285 | [6OSO3]Galb1-4[6OSO3]GlcNAcb-Sp0 | 46 | 6 |
| 401 | Gala1-4Galb1-4GlcNacb1-2Mana1-3(Gala1-4Galb1-4GlcNacb1-2Mana1-6)Manb1-4GlcNacb1-4GlcNa [symbol] | 46 | 4 |
| 379 | Galb1-3GlcNacb1-3(Galb1-3GlcNacb1-3Galb1-4GlcNacb1-6)Galb1-4Glcb-Sp0 | 46 | 22 |
| 284 | [3OSO3][4OSO3]Galb1-4GlcNAcb-Sp0 | 42 | 8 |
| 131 | Galb1-3GlcNAcb1-3Galb1-4Glcb-Sp10 | 42 | 12 |
| 327 | Gala1-4Galb1-4GlcNAcb1-3Galb1-4Glcb-Sp0 | 41 | 15 |
| 31 | [3OSO3]Galb1-3GlcNAcb-Sp8 | 40 | 5 |
| 265 | [3OSO3]Galb1-4(Fuca1-3)GlcNAc-Sp0 | 40 | 11 |
| 133 | Galb1-3GlcNAcb-Sp8 | 39 | 12 |
| 12 | Neu5Aca-Sp8 | 39 | 9 |
| 357 | Fuca1-2Galb1-4(Fuca1-3)GlcNAcb1-2Mana1-3(Fuca1-2Galb1-4(Fuca1-3)GlcNAcb1-2Mana1-6)Manb1 [symbol] | 38 | 22 |
| 3 | Neu5Gcb2-6Galb1-4GlcNAc-Sp8 | 37 | 7 |
| 269 | Fuca1-2[6OSO3]Galb1-4Glc-Sp0 | 37 | 14 |
| 283 | [3OSO3]Galb1-4[6OSO3]GlcNAcb-Sp0 | 37 | 14 |
| 162 | GlcNAcb1-3Galb1-3GalNAca-Sp8 | 37 | 18 |
| 78 | GalNAca1-3(Fuca1-2)Galb1-3GlcNAcb-Sp0 | 36 | 11 |
| 8 | GalNAca-Sp8 | 36 | 15 |
| 249 | Neu5Aca2-6Galb-Sp8 | 36 | 12 |
| 94 | Gala1-3(Fuca1-2)Galb1-3GlcNAcb-Sp0 | 36 | 13 |
| 138 | Galb1-4[6OSO3]Glcb-Sp0 | 36 | 23 |
| 93 | Gala1-2Galb-Sp8 | 36 | 16 |
| 139 | Galb1-4[6OSO3]Glcb-Sp8 | 35 | 10 |
| 268 | Fuca1-2[6OSO3]Galb1-4[6OSO3]Glc-Sp0 | 35 | 19 |
| 9 | Fuca-Sp8 | 34 | 19 |
| 62 | Fuca1-2Galb1-3GlcNAcb1-3Galb1-4Glcb-Sp8 | 34 | 5 |
| 264 | [3OSO3]Galb1-4(Fuca1-3)[6OSO3]GlcNAc-Sp8 | 33 | 9 |
| 271 | Galb1-3(Fuca1-4)GlcNAcb1-3Galb1-3(Fuca1-4)GlcNAcb-Sp0 | 32 | 14 |
| 116 | Galb1-3(Fuca1-4)GlcNAcb-Sp0 | 32 | 5 |
| 278 | Galb1-4(Fuca1-3)[6OSO3]Glc-Sp0 | 32 | 8 |
| 318 | Neu5Aca2-6Galb1-4GlcNAcb1-2Mana1-3(Neu5Aca2-6Galb1-4GlcNAcb1-2Mana1-6)Manb1-4GlcNAcb [symbol] | 32 | 14 |
| 104 | Gala1-3Galb1-4GlcNAcb-Sp8 | 32 | 12 |
| 258 | Neu5Gca2-3Galb1-4Glcb-Sp0 | 31 | 12 |
| 175 | GlcNAcb1-6Galb1-4GlcNAcb-Sp8 | 31 | 7 |
| 82 | GalNAca1-3(Fuca1-2)Galb1-4Glcb-Sp0 | 31 | 18 |
| 66 | Fuca1-2Galb1-4(Fuca1-3)GlcNAcb1-3Galb1-4(Fuca1-3)GlcNAcb1-3Galb1-4(Fuca1-3)GlcNAcb-Sp0 | 31 | 13 |
| 70 | Fuca1-2Galb1-4GlcNAcb1-3Galb1-4GlcNAcb1-3Galb1-4GlcNAcb-Sp0 | 31 | 9 |
| 99 | Gala1-3(Gala1-4)Galb1-4GlcNAcb-Sp8 | 31 | 14 |
| 118 | Galb1-3(Fuca1-4)GlcNAcb-Sp8 | 30 | 5 |
| 95 | Gala1-3(Fuca1-2)Galb1-4(Fuca1-3)GlcNAcb-Sp0 | 30 | 24 |
| 213 | Neu5Aca2-3GalNAca-Sp8 | 30 | 7 |
| 189 | Mana1-2Mana1-3(Mana1-2Mana1-6)Mana-Sp9 | 30 | 22 |

50 glycan structures giving the highest signals out of the 442 glycan structures on the microarray are shown. [symbol]
indicates data missing or illegible when filed

TABLE 5
Glycan binding specificity of Fab1.4.24.

| Chart # | Masterlist Name | RFU | STDEV |
|---|---|---|---|
| 200 | Fuca1-3(Galb1-4)GlcNAcb1-2Mana1-3(Fuca1-3(Galb1-4)GlcNAcb1-2Mana1-6)Manb1-4GlcNAcb1-4G [symbol] | 7329 | 784 |
| 283 | [3OSO3]Galb1-4[6OSO3]GlcNAcb-Sp0 | 5285 | 1197 |
| 183 | GlcAb-Sp8 | 4868 | 2337 |
| 387 | Fuca1-2Galb1-3GalNAca1-3(Fuca1-2)Galb1-4GlcNAcb-Sp0 | 4789 | 1149 |
| 193 | Mana1-2Mana1-2Mana1-3(Mana1-2Mana1-3(Mana1-2Mana1-6)Mana1-6)Manb1-4GlcNAcb1-4GlcNA [symbol] | 4749 | 281 |
| 185 | GlcAb1-6Galb-Sp8 | 4669 | 601 |
| 13 | Neu5Aca-Sp11 | 4621 | 1095 |
| 44 | Neu5Aca2-3[6OSO3]Galb1-4GlcNAcb-Sp8 | 4365 | 1313 |
| 206 | Neu5Aca2-8Neu5Aca2-8Neu5Aca-Sp8 | 4098 | 1333 |
| 256 | Neu5Gca2-3Galb1-4(Fuca1-3)GlcNAcb-Sp0 | 4072 | 551 |
| 25 | [3OSO3][6OSO3]Galb1-4GlcNAcb-Sp0 | 4032 | 487 |
| 204 | Neu5Aca2-8Neu5Aca2-8Neu5Aca2-3Galb1-4Glcb-Sp0 | 3951 | 609 |
| 43 | [6OSO3]Galb1-4[6OSO3]Glcb-Sp8 | 3945 | 1008 |
| 371 | NeuAca2-3Galb1-4GlcNAcb1-3GalNAc-Sp14 | 3923 | 762 |
| 288 | Gala1-3GalNAca-Sp16 | 3896 | 349 |
| 145 | Galb1-4GlcNAcb1-3Galb1-4GlcNAcb1-3Galb1-4GlcNAcb-Sp0 | 3798 | 1148 |
| 21 | GlcN(Gc)b-Sp8 | 3597 | 2167 |
| 76 | Fuca1-4GlcNAcb-Sp8 | 3591 | 1328 |
| 147 | Galb1-4GlcNAcb1-3Galb1-4Glcb-Sp0 | 3589 | 1537 |
| 220 | Neu5Aca2-3Galb1-3(Neu5Aca2-6)GalNAca-Sp8 | 3485 | 296 |
| 181 | G-ol-Sp8 | 3482 | 740 |
| 112 | Gala1-6Glcb-Sp8 | 3346 | 1231 |
| 24 | [3OSO3][6OSO3]Galb1-4[6OSO3]GlcNAcb-Sp0 | 3281 | 449 |
| 139 | Galb1-4[6OSO3]Glcb-Sp8 | 3275 | 1017 |
| 333 | GlcNAca1-4Galb1-4GlcNAcb-Sp0 | 3204 | 1030 |
| 171 | (GlcNAcb1-4)5b-Sp8 | 3187 | 745 |
| 265 | [3OSO3]Galb1-4(Fuca1-3)GlcNAc-Sp0 | 3183 | 970 |
| 285 | [6OSO3]Galb1-4[6OSO3]GlcNAcb-Sp0 | 3120 | 1553 |
| 36 | [3OSO3]Galb-Sp8 | 3098 | 1195 |
| 172 | GlcNAcb1-4GlcNAcb1-4GlcNAcb-Sp8 | 3078 | 956 |
| 55 | Fuca1-2Galb1-SGalNAcb1-3Gala-Sp9 | 3078 | 1358 |
| 233 | Neu5Aca2-3Galb1-4GlcNAcb1-3Galb1-4(Fuca1-3)GlcNAc-Sp0 | 3040 | 637 |
| 78 | GalNAca1-3(Fuca1-2)Galb1-3GlcNAcb-Sp0 | 2992 | 802 |
| 359 | Mana1-3(Galb1-4GlcNAcb1-2Mana1-6)Manb1-4GlcNAcb1-4GlcNAcb-Sp12 | 2970 | 545 |
| 137 | Galb1-4(Fuca1-3)GlcNAcb1-4Galb1-4(Fuca1-3)GlcNAcb1-4Galb1-4(Fuca1-3)GlcNAcb-Sp0 | 2926 | 1841 |
| 373 | Neu5Aca2-3Galb1-4(Fuca1-3)GlcNAcb1-3GalNAca-Sp14 | 2867 | 589 |
| 33 | [3OSO3]Galb1-4[6OSO3]GlcNAcb-Sp8 | 2790 | 634 |
| 184 | GlcAb1-3Galb-Sp8 | 2772 | 230 |
| 258 | Neu5Gca2-3Galb1-4Glcb-Sp0 | 2768 | 255 |
| 286 | 6-H2PO3Glcb-Sp10 | 2728 | 161 |
| 30 | [3OSO3]Galb1-3GalNAca-Sp8 | 2688 | 378 |
| 345 | Galb1-4GlcNAcb1-2Mana1-3(Mana1-6)Manb1-4GlcNAcb1-4GlcNAcb-Sp12 | 2673 | 669 |
| 35 | [3OSO3]Galb1-4GlcNAcb-Sp8 | 2665 | 462 |
| 254 | Neu5Gca2-3Galb1-3(Fuca1-4)GlcNAcb-Sp0 | 2652 | 470 |
| 197 | Mana1-6(Mana1-3)Mana1-6(Mana1-3)Manb1-4GlcNAcb1-4 GlcNAcb-Sp12 | 2636 | 1504 |
| 284 | [3OSO3][4OSO3]Galb1-4GlcNAcb-Sp0 | 2611 | 122 |
| 14 | Neu5Acb-Sp8 | 2589 | 428 |
| 37 | [4OSO3][6OSO3]Galb1-4GlcNAcb-Sp0 | 2585 | 414 |
| 323 | Neu5Ac(9Ac)a2-3Galb1-3GlcNAcb-Sp0 | 2550 | 875 |
| 243 | Neu5Aca2-6Galb1-4GlcNAcb-Sp0 | 2527 | 579 |

50 glycan structures giving the highest signals out of the 442 glycan structures on the microarray are shown. [symbol]
indicates data missing or illegible when filed

TABLE 6
Glycan binding specificity of anti-sialyl Lewis a KM231.

| Iupac | Average Signal |
|---|---|
| NeuAca2-3Galb1-3GlcNAcb#Sp8 | 50939.82 |
| NeuAca2-3Gal[6S]b1-3GlcNAc#Sp8 | 49372.617 |
| NeuAca2-3Galb1-3GlcNAcb#Sp0 | 49296.074 |
| NeuAca2-3Galb1-3GlcNAcb1-3Galb1-3GlcNAcb#sp0 | 47685.07 |
| NeuAca2-3Galb1-3GlcNAcb1-3Galb1-4GlcNAcb#Sp0 | 47434.13 |
| NeuAca2-3Galb1-3GalNAcb1-3Gala1-4Galb1-4Glcb#Sp0 | 45506.75 |
| NeuAca2-3Galb1-4(Fuca1-3)GlcNAc[6S]b#Sp8 | 45451.79 |
| NeuAca2-3Galb1-4(Fuca1-3)GlcNAc[6S]b#Sp8 | 45379.73 |
| NeuAca2-3Galb1-3GlcNAcb#Sp8 | 44223.01 |
| NeuAca2-3Galb1-3GlcNAcb1-3Galb1-4GlcNAcb#Sp0 | 44197.83 |
| NeuAca2-3Galb1-3(NeuAca2-3Galb1-4)GlcNAcb#Sp8 | 44142.84 |
| NeuAca2-3Galb1-3(Fuca1-4)GlcNAcb1-3Galb1-4(Fucal-3)GlcNAcb#Sp0 | 44027.2 |
| NeuAca2-3Galb1-3(Fuca1-4)GlcNAcb#Sp8 | 43964.05 |
| NeuAca2-3Galb1-3(NeuAca2-3Galb1-4)GlcNAcb#Sp8 | 43231.15 |
| NeuGca2-3Galb1-3GlcNAcb#Sp0 | 41986.797 |
| NeuGca2-3Galb1-3(Fuca1-4)GlcNAcb#Sp0 | 41924.15 |
| Galb1-3(Fuca1-4)GlcNAc#Sp0 | 41621.58 |
| Fuca1-2Galb1-3(Fuca1-4)GlcNAcb#Sp8 | 41112.434 |
| NeuGca2-3Galb1-3GlcNAcb#Sp0 | 40815.547 |
| NeuAca2-3Galb1-3(Fuca1-4)GlcNAcb1-3Galb1-4(Fuca1-3)GlcNAcb#Sp0 | 39077.062 |
| NeuAca2-3Galb1-3GlcNAcb#Sp0 | 38127.22 |
| NeuAca2-3Galb1-3(Fuca1-4)GlcNAcb#Sp8 | 37920.363 |
| NeuAca2-3Galb1-3GlcNAcb1-3Galb1-3GlcNAcb#Sp0 | 37777.668 |
| NeuGca2-3Galb1-3(Fuca1-4)GlcNAcb#Sp0 | 36370.31 |
| Galb1-3(Fuca1-4)GlcNAcb#Sp8 | 35107.07 |
| Galb1-3(Fuca1-4)GlcNAc#Sp8 | 33333.863 |
| Galb1-3(Fuca1-4)GlcNAcb#Sp8 | 32780.67 |
| NeuAca2-3Galb1-3GalNAcb1-3Gala1-4Galb1-4Glcb#Sp0 | 31469.928 |
| Fuca1-2Galb1-3(Fuca1-4)GlcNAcb#SD8 | 29192.773 |
| Galb1-3(Fuca1-4)GlcNAc#Sp8 | 28119.258 |
| Galb1-3(Fuca1-4)GlcNAcb1-3Galb1-4(Fuca1-3)GlcNAcb# Sp0 | 23256.559 |
| Galb1-3(Fuca1-4)GlcNAc#Sp0 | 21807.383 |
| NeuAca2-3Galb1-3GalNAca#Sp8 | 21617.492 |
| Galb1-3(Fuca1-4)GlcNAcb1-3Galb1-3(Fuca1-4)GlcNAcb# sp0 | 18689.695 |
| NeuAca2-3Galb1-3GalNAc[6S]a#Sp8 | 13858.561 |
| Galb1-3(Fuca1-4)GlcNAcb1-3Galb1-4(Fuca1-3)GlcNAcb# Sp0 | 12142.388 |
| NeuAca2-6Galb1-4GlcNAcb1-3Galb1-4(Fuca1-3)GlcNAcb1 - | 10530.401 |
| 3Galb1-4(Fuca1-3)GlcNAcb#Sp0 | |

38 glycan structures giving the highest signals are shown.

TABLE 7
Common structural categories among the glycan epitopes defining a cryptic
subpopulation of mesenchymal stem cells. The antibodies that bind to each category are indicated.

| | Anti-GT1b | Anti-Tn | Tra-1-60 | Tra-1-81 | Fab 1.4.24 | Anti-sLea |
|---|---|---|---|---|---|---|
| 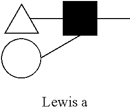 | + | + | | + | + | + |
| 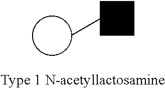 | + | + | + | + | | |
| 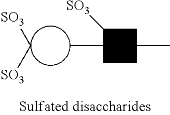 | + | + | | | + | + |
| 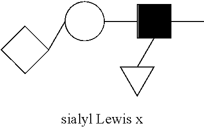 | + | + | | | + | + |
| 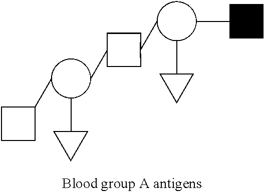 | A1 | | | A1 | A2 | |
| 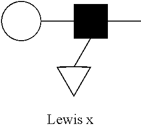 | + | + | | | | + |
| 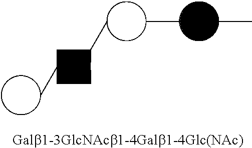 | | + | + | + | | |

TABLE 8
Preferred terminal H-type II and type I N-acetyllactosamine epitopes

| | | CB | BM | adipo | osteo | chondro |
|---|---|---|---|---|---|---|
| Trivial name | Terminal epitope | MSC | MSC | diff. | diff. | diff. |
| LN type 1, Lec | Galβ3GlcNAcβ | + | + | + | +/− | q |
| | | L+ | L+ | Lq | L+ | Lq |
| | Lecβ3Galβ4Glc[NAc]β | +/− | +/− | q | +/− | q |
| Lea | Galβ3(Fucα4)GlcNAcβ | + | + | ++ | + | |
| | | L+/− | L+/− | | L+/− | |
| | Leaβ3Galβ4Glc[NAc]β | +/− | +/− | | +/− | |
| sialyl Lea, sLea | SAα3Galβ3(Fucα4)GlcNAcβ | +/− | +/− | ++ | + | |
| | | L+ | L+ | | L+ | |
| | sLeaβ3Galβ4Glc[NAc]β | +/− | +/− | | +/− | |
| α3′-sialyl Lec | SAα3Galβ3GlcNAcβ | +/− | +/− | ++ | + | q |
| | | Lq | Lq | Lq | Lq | |
| H type 2, H2 | Fucα2Galβ4GlcNAcβ | + | +/− | ++ | + | q |
| | | L+ | L+ | Nq | L+ | |
| | | Nq | Nq | | Nq | |
| | H2p2Manα3/6 | q | q | q | q | |
| | H2p3Galβ4Glc[NAc]β | + | + | | + | |

1) Stem cell and differentiated cell types are abbreviated as in other parts of the present document; CB/BM indicates MSC derived from cord blood or bone marrow; adipo/osteo/chondro diff. indicates cells differentiated into adipocyte, osteoblast, or chondrocyte direction from MSC.

2) Occurrence of terminal epitopes in glycoconjugates and/or specifically in N-glycans (N), O-glycans (O), and/or glycosphingolipids (L). Code: q, qualitative data; +/−, low expression; +, common; ++, abundant.

1.-31. (canceled)

32. A method of targeting of a human stem cell population involving a step of binding a specific glycan binder reagent to a cryptic glycan epitope on necrotic cells, wherein the cryptic glycan epitope is exposed under cell damaging conditions and wherein the glycan binder reagent binds to cryptic glycan epitope structures comprising common core Galβ3/4HexNAc or GalNAcα, preferably Galβ3/4GlcNAc, which may further include sialic acid and/or fucose and/or sulphate.

33. The method according to claim 32, wherein the targeting is quality control of a cell population.

34. The method according to claim 32 for the evaluation of cells as a quality control step in a cell production process and/or verification of cell status with regard to harmful conditions potentially causing the exposure of the novel cryptic epitopes.

35. The method according to any of claims 32, wherein the cryptic glycan epitope is a N-acetyllactosamine structure according to Formula
[Mα]ₘGalβ1-3/4[Nα]ₙGlcNAcβxHex
wherein x is linkage position 2, or 3, wherein m, and n are integers 0, or 1, independently; and
Hex is Man and βxHex is β2Man, or Hex is Gal and βxHex is β3Gal;
M and N are monosaccharide residues being i) independently nothing (free hydroxyl groups at the positions) and/or ii) SA, which is Sialic acid linked to the 3-position of Gal and/or iii) Fuc (L-fucose) residue linked to the 2-position of Gal and/or 4 position of GlcNAc, with the provision, that when Gal is linked to position 4 of GlcNAc, N is nothing and M is Fucα2 and the structure is H type II or M is SAα3/6, preferably SAα3 and the structure is keratan sulphate structure; that when Gal is linked to the position 3 of GlcNAc, and the structure is type I N-acetyllactosamine so that N is nothing or Fucα4 and M is nothing or SAα3.

36. The method according to claim 32, wherein the binder reagent binds to type I N-acetyllactosamines comprising the core epitope Galβ3GlcNAc and its fucosylated and/or sialylated derivatives, including Lewis a, sialyl-Lewis a, large multisialylated ganglioseries gangliosides, type II N-acetyllactosamines including H-type II antigen, keratan sulphate epitopes, or small mucin type O-glycans, including Tn and sialyl Tn antigens.

37. The method according to claim 32, wherein the binder reagent or reagents are selected from the list of: anti-Lewis c, anti-H type 2, anti-Lewis a, anti-sialyl Lewis a, anti-GT1b, anti-GQ1b, anti-keratan sulfate Tra-1-81 or Tra-1-60, anti-Tn, anti-sialyl Tnl and Fab-fragment Fab1.4.24.

38. The method according to claim 32, wherein the binder reagent binds to sialic acid containing and sialidase sensitive epitopes recognized by the Fab-fragment Fab1.4.24.

39. The method according to claim 32, wherein the cells are mesenchymal stem cells or cells differentiated thereof.

40. The method according to claim 32, wherein the cryptic epitope is revealed under necrotic cell conditions selected from the group:
a. exposure to a toxic agent,
b. exposure to lipophilic agents,
c. exposure to harmful temperature conditions

41. The method according to claim 40, wherein the cryptic epitope is revealed under necrotic cell conditions selected from the group:
a. exposure to a toxic cation chelating agent, most preferably EDTA;
b. exposure to lipophilic detergents, preferably Triton X-100;
c. exposure to temperatures of above +38 or below +35° C.

42. The method according to claim 40, wherein the cryptic epitopes of cells are revealed under conditions that maintain integrity of the cells sufficient enough for analysis.

43. The method according to claim 40, wherein condition includes exposure to about 1-10 mM EDTA; 0.1% Triton X-100; or heating condition equivalent of about 56° C. for 30 min.

44. The method according to claim 40, including the following further steps:
d. Contacting the cell preparation with said binder reagent for the novel cryptic epitope after the exposure to the conditions damaging the cell membranes and exposing the cryptic epitopes;
e. Measuring the binding of the binder molecule to the cryptic epitope.

45. The method according to claim 32 for the purpose selected from the group:
i) the analysis of damage caused by cell detachment method or passaging process;
ii) assessing the extent of damage to cells caused by growth conditions or handling, for example detachment from culture plates or exposure to varying thermal conditions e.g. during freezing and thawing or by exposure of toxic or lipophilic compounds during cell growth/culture or handling;
iii) for the separation of the damaged cells from non-damaged cells, preferably by cell sorting or immobilization methods using the binder reagents;
iv) for analyzing cells with regard to the cryptic epitopes.

46. The method according to claim 32, wherein part of the cell population reveals the cryptic glycan under “natural type conditions” including regular cell handling and in vivo physiological conditions.

47. The method according to claim 32, wherein the targeting is evaluation, preferably quality control, and/or manipulation, preferably sorting, of a cell population.

48. The method according to claim 32, wherein cell labeling in the range of 1-50% of the cell population is observed.

49. The method according to claim 32, wherein cell labeling in the range of 51-100% of the cell population is observed.

50. The method according to claim 32, wherein the damaged cells are separated from non-damaged cells and non-damaged cells by a binder for cryptic glycan epitopes and the non-damaged cells are cultivated.

51. A method to evaluate the presence of the novel cryptic markers in cells including the following steps:
a. Exposing a cell preparation or its fraction to conditions damaging the cell membranes and exposing the cryptic epitopes;
b. Contacting the cell preparation with binder reagent for the novel cryptic epitope after the exposure to the conditions damaging the cell membranes and exposing the cryptic epitopes, wherein the glycan binder reagent binds to cryptic glycan epitope structures; comprising common core Galβ3/4HexNAc or GalNAcβ, preferably Galβ3/4GlcNAc, which may further include sialic acid and/or fucose and/or sulphate
c. Measuring the binding of the binder molecule to the cryptic epitope.
